SEQUENCE LISTING (17 sequences)

SEQ ID NO: 1 - DNA, 2870 bp, Corynebacterium glutamicum
  source 1..2870: mol_type = genomic DNA
  CDS 78..2837: note = Phosphoenolpyruvate_carboxylase_coding_region
aaacacctgttttgctgggtgattttttatctcatgcacgccaacaccctcaatgtgaaagagtgtttaaagtagttatgactgattttttacgcgatgacatcaggttcctcggtcaaatcctcggtgaggtaattgcggaacaagaaggccaggaggtttatgaactggtcgaacaagcgcgcctgacttcttttgatatcgccaagggcaacgccgaaatggatagcctggttcaggttttcgacggcattactccagccaaggcaacaccgattgctcgcgcattttcccacttcgctctgctggctaacctggcggaagacctctacgatgaagagcttcgtgaacaggctctcgatgcaggcgacacccctccggacagcactcttgatgccacctggctgaaactcaatgagggcaatgttggcgcagaagctgtggccgatgtgctgcgcaatgctgaggtggcgccggttctgactgcgcacccaactgagactcgccgccgcactgtttttgatgcgcaaaagtggatcaccacccacatgcgtgaacgccacgctttgcagtctgcggagcctaccgctcgtacgcaaagcaagttggatgagatcgagaagaacatccgccgtcgcatcaccattttgtggcagaccgcgttgattcgtgtggcccgcccacgtatcgaggacgagatcgaagtagggctgcgctactacaagctgagccttttggaagagattccacgtatcaaccgtgatgtggctgttgagcttcgtgagcgtttcggcgagggtgttcctttgaagcccgtggtcaagccaggttcctggattggtggagaccacgacggtaacccttatgtcaccgcggaaacagttgagtattccactcaccgcgctgcggaaaccgtgctcaagtactatgcacgccagctgcattccctcgagcatgagctcagcctgtcggaccgcatgaataaggtcaccccgcagctgcttgcgctggcagatgcagggcacaacgacgtgccaagccgcgtggatgagccttatcgacgcgccgtccatggcgttcgcggacgtatcctcgcgacgacggccgagctgatcggcgaggacgccgttgagggcgtgtggttcaaggtctttactccatacgcatctccggaagaattcttaaacgatgcgttgaccattgatcattctctgcgtgaatccaaggacgttctcattgccgatgatcgtttgtctgtgctgatttctgccatcgagagctttggattcaacctttacgcactggatctgcgccaaaactccgaaagctacgaggacgtcctcaccgagcttttcgaacgcgcccaagtcaccgcaaactaccgcgagctgtctgaagcagagaagcttgaggtgctgctgaaggaactgcgcagccctcgtccgctgatcccgcacggttcagatgaatacagcgaggtcaccgaccgcgagctcggcatcttccgcaccgcgtcggaggctgttaagaaattcgggccacggatggtgcctcactgcatcatctccatggcatcatcggtcaccgatgtgctcgagccgatggtgttgctcaaggaattcggactcatcgcagccaacggcgacaacccacgcggcaccgtcgatgtcatcccactgttcgaaaccatcgaagatctccaggccggcgccggaatcctcgacgaactgtggaaaattgatctctaccgcaactacctcctgcagcgcgacaacgtccaggaagtcatgctcggttactccgattccaacaaggatggcggatatttctccgcaaactgggcgctttacgacgcggaactgcagctcgtcgaactatgccgatcagccggggtcaagcttcgcctgttccacggccgtggtggcaccgtcggccgcggtggcggaccttcctacgacgcgattcttgcccagcccaggggggctgtccaaggttccgtgcgcatcaccgagcagggcgagatcatctccgctaagtacggcaaccccgaaaccgcgcgccgaaacctcgaagccctggtctcagccacgcttgaggcatcgcttctcgacgtctccgaactcaccgatcaccaacgcgcgtacgacatcatgagtgagatctctgagctcagcttgaagaagtacgcctccttggtgcacgaggatcaaggcttcatcgattacttcacccagtccacgccgctgcaggagattggatccctcaacatcggatccaggccttcctcacgcaagcagacctcctcggtggaagatttgcgagccatcccatgggtgctcagctggtcacagtctcgtgtcatgctgccaggctggtttggtgtcggaaccgcattagagcagtggattggcgaaggggagcaggccacccaacgcattgccgagctgcaaacactcaatgagtcctggccatttttcacctcagtgttggataacatggctcaggtgatgtccaaggcagagctgcgtttggcaaagctctacgcagacctgatcccagatacggaagtagccgagcgagtctattccgtcatccgcgaggagtacttcctgaccaagaagatgttctgcgtaatcaccggctctgatgatctgcttgatgacaacccacttctcgcacgctctgtccagcgccgatacccctacctgcttccactcaacgtgatccaggtagagatgatgcgacgctaccgaaaaggcgaccaaagcgagcaagtgtcccgcaacattcagctgaccatgaacggtctttccactgcgctgcgcaactccggctagtccagccggctgggtagtactcgtgtatactgt

SEQ ID NO: 2 - protein, 919 aa, Corynebacterium glutamicum
MTDFLRDDIRFLGQILGEVIAEQEGQEVYELVEQARLTSFDIAKGNAEMDSLVQVFDGITPAKATPIARAFSHFALLANLAEDLYDEELREQALDAGDTPPDSTLDATWLKLNEGNVGAEAVADVLRNAEVAPVLTAHPTETRRRTVFDAQKWITTHMRERHALQSAEPTARTQSKLDEIEKNIRRRITILWQTALIRVARPRIEDEIEVGLRYYKLSLLEEIPRINRDVAVELRERFGEGVPLKPVVKPGSWIGGDHDGNPYVTAETVEYSTHRAAETVLKYYARQLHSLEHELSLSDRMNKVTPQLLALADAGHNDVPSRVDEPYRRAVHGVRGRILATTAELIGEDAVEGVWFKVFTPYASPEEFLNDALTIDHSLRESKDVLIADDRLSVLISAIESFGFNLYALDLRQNSESYEDVLTELFERAQVTANYRELSEAEKLEVLLKELRSPRPLIPHGSDEYSEVTDRELGIFRTASEAVKKFGPRMVPHCIISMASSVTDVLEPMVLLKEFGLIAANGDNPRGTVDVIPLFETIEDLQAGAGILDELWKIDLYRNYLLQRDNVQEVMLGYSDSNKDGGYFSANWALYDAELQLVELCRSAGVKLRLFHGRGGTVGRGGGPSYDAILAQPRGAVQGSVRITEQGEIISAKYGNPETARRNLEALVSATLEASLLDVSELTDHQRAYDIMSEISELSLKKYASLVHEDQGFIDYFTQSTPLQEIGSLNIGSRPSSRKQTSSVEDLRAIPWVLSWSQSRVMLPGWFGVGTALEQWIGEGEQATQRIAELQTLNESWPFFTSVLDNMAQVMSKAELRLAKLYADLIPDTEVAERVYSVIREEYFLTKKMFCVITGSDDLLDDNPLLARSVQRRYPYLLPLNVIQVEMMRRYRKGDQSEQVSRNIQLTMNGLSTALRNSG

SEQ ID NO: 3 - protein, 919 aa, Corynebacterium efficiens
MNELLRDDIRYLGRILGEVISEQEGHHVFELVERARRTSFDIAKGRAEMDSLVEVFAGIDPEDATPVARAFTHFALLANLAEDLHDAAQREQALNSGEPAPDSTLEATWVKLDDAGVGSGEVAAVIRNALVAPVLTAHPTETRRRTVFDAQKHITALMEERHLLLALPTHARTQSKLDDIERNIRRRITILWQTALIRVARPRIEDEVEVGLRYYKLSLLAEIPRINHDVTVELARRFGGDIPTTAMVRPGSWIGGDHDGNPFVTAETVTYATHRAAETVLKYYVKQLHALEHELSLSDRMNVISDELRVLADAGQNDMPSRVDEPYRRAIHGMRGRMLATTAALIGEEAVEGTWFKTFTPYTDTHEFKRDLDIVDGSLRMSRDDIIADDRLAMLRSALDSFGFNLYSLDLRQNSDGFEDVLTELFATAQTEKNYRGLTEAEKLDLLIRELSTPRPLIPHGDPDYSEATNRELGIFSKAAEAVRKFGPLMVPHCIISMASSVTDILEPMVLLKEFGLIRANGKNPTGSVDVIPLFETIDDLQRGAGILEELWDIDLYRNYLEQRDNVQEVMLGYSDSNKDGGYFAANWALYDAELRLVELCRGRNVKLRLFHGRGGTVGRGGGPSYDAILAQPKGAVRGAVRVTEQGEIISAKYGNPDTARRNLEALVSATLEASLLDDVELPNRERAHQIMGEISELSFRRYSSLVHEDPGFIQYFTQSTPLQEIGSLNIGSRPSSRKQTNTVEDLRAIPWVLSWSQSRVMLPGWFGVGTALREWIGEGEGAAERIAELQELNRCWPFFTSVLDNMAQVMSKAELRLARLYADLIPDREVADRIYETIFGEYFLTKEMFCTITGSQDLLDDNPALARSVRSRFPYLLPLNVIQVEMMRRYRSGDEGTAVPRNIRLTMNGLSTALRNSG

SEQ ID NO: 4 - protein, 919 aa, Corynebacterium callunae
MNDLLRDDIRFLGRILGKVISEQEGSEVYELVERARQTSFQIAKGNLEMDTLVEVFKGIDPEKATPVARAFTHFALLANLAEDLHDAEAREQALDSGETPPESTLESTWLKLDEAEVKASDVSDVLRNAQVAPVLTAHPTETRRRTVFDAQKWITAYMQERQLLLAAPKNARTQAKLDAIEKNIHRRISVLWQTALIRVARPRIEDEIEVGLRYYKLSLLEEIPRINRDVVLELRSRYGKVIPAKAVIKPGSWIGGDHDGNPYVTADVVAYSTSRAAETVLKYYGRQLHTLEHELSLSDRMSSVTEELRELADAGKNDVPSRVDEPYRRAVHGIRGRILATTAHLIGEHAVEGTWFKIFEPYSSPEEFAAELKIVDDSLRASHDELIADDRLAAIYAAVQSFGFNLYSLDLRQNSESYEDVLTELFQNAQVTADYRNLDEAAKTELLLKELRSPRPLIPNGGWDFTEPTERELGIFKMAAHAVEKFGPNMVPHCIISMASSVTDVLEPMVLLKEFGLIKAKGDTPVGSIDVIPLFETIDDLQAGAGILEDLWKIDLYRNYLQQRDNVQEVMLGYSDSNKDGGYFSANWALYDAELQLVDLCRSAGVKLRLFHGRGGTVGRGGGPSYDAILAQPKGAVRGSLRITEQGEIISAKYGSPETARRNLEALVSATLEASLLDVSDLSDPERAYEIMREISELSLQKYSSLVHEDPGFIDYFTQSTPLREIGSLNIGSRPSSRKQTSSVEDLRAIPWVLSWSQSRVMLPGWFGVGTALQQWIGSGEQATERIKELQELNETWPFFTSVLDNMAQVMSKAELRLAKLYSELIPDREVAQRIYDVIFDEYFLTKEMFCVITGSTDLLDENPLLARSVRSRFPYLLPLNVIQVEMMRRYRAGDESKGVSRNIQLTMNGLATALRNSG

SEQ ID NO: 5 - protein, 920 aa, Corynebacterium ammoniagenes
MSEQVRDDIRLLGRILGQVIAEQEGEDVYELVESTRRLAFGVARGEEDAEALLSAFRAVDENKINLVARSFSHFALMANIAEDLDDESALAAREDEGAPAPDASLEAVLAKLQAAGDISTSDITRVLDTAQVSPVFTAHPTETRRRTVFDVQARIIALLRERHGILAQPETTRRKARLAEIEREAHLRMTILWQTALIRIARPQIEDEANVGLRYFKRSLLEQVPAINRDTIAGLREAFGSAVPNRQVVRTGSWIGGDHDGNPYVTGETLRYATRQAADTVLEYYVDELALLEKELSLSDRYSESSAELQELAARGNNDVPSRVDEPYRRAIHGVHGRMVATRAAIANTQSDTEQGEFAPYASPSEFAADLSVIDRSLRQFNDAIIAEDRLLRIRSAVDTFGFHLNALDLRQNSESFEFVLDELFAAAGVTAAGAGYKDLDENAKRELLIAELTSARPLTFGWSKGFSETTERELGIFRAAAEAINDLGPEVVPHCIVSMTGTVSDILEPMVLLKEVGIISFDPAQQRLVGSVDIAPLFETIEDLQAGARILEELWEVDLYRHYLRGRNDTQEVVLGYSDSNKDGGYLLANWALYDAQIDIVDACERHGVALRFSHGRGGVVGRGGGPTYDAILAQPEGAVRGSVRITEQGEVISARYGTATSARRHLEAFVAGTLEASLLDTERLKQPERAYDIMREVSSLAGEKYKQLVRDDEGFIDYFTQSTPLHEIGDLNLGSRPTARKQTESISDLRAIPWVLSWSQSRVNLPGWFGVGSGITQWAGEDDQRWDDLRTLYQAWPFFRSVLDNMAQVMGKASMDLAKIYSTLVDDAETSKRVFTTIVDEYELTREVFHRITGHESLMAGNERLERSVHRRYPYLLPLNAIQIELLRRYRAGDDSFLVSKTIQVTMNGLATGLRTSG

SEQ ID NO: 6 - protein, 923 aa, Corynebacterium marinum
MADRDHLQEDIRYLGRILGRVIAEQEGEDVFDLVEHARQQAFEVARENASLEVLVELFRNIDPARATPVIRAFSHFALMANLAEDIHDDFNRERILDEGGVPASTLEATWEKFEAAGVSPADVEKVVSDALVAPVFTAHPTETRRRTVFDAQKKITGLMLTRHQLQDAETTARTQERLDDIERSIRRRMTILWQTALIRQARPRIEDEIEVGLRYYHLSLLREIPALNRAVHDTLTSRFDVRFRDGGGSAIVRPGSWIGGDHDGNPFVTADTLDYASRRAAQTVLKHYSGELHALEHELSLSDRMTSVSVELVGLAARGRNDVPSRVDEPYRQAIHGIRGRILATTAALIGEDAVEGVWHREHQPYASAAEFDADLRIVDESLRSSNDAIIADDCLAAIRAAVASFGFHLHSIDLRQNSESFENVLTEVFATAHVHPGYSALGEEEKIELLIGELRTPRPLVPRGYRGFSEATQRELDLLNRAADSVAHFGAEMIPHQIISMAQSVSDILEPMVLLKEVGLIRANGAGPTGSVDIIPLFETIDDLEAGAGILRRLWQLPLYQHYLEHRGNVQEVMLGYSDSNKDGGYFAANWALYDAETQLVQAGRDHGVRLRLFHGRGGTVGRGGGPSYEAILAQPQGAVDGSVRITEQGEIISAKYGSPRTARRNLEALVSATLEASLLPVDELTDRDRAMEIMRELSLISRRKYSQLVHDDPGFIRYFTQSTPLDEIGSLNIGSRPTSRKQTTAVEDLRAIPWVLAWSQSRVLLPGWFGVGTALEEWIGQGPDRADRVAELRALYESWPFFASVMSNMAQVMSKAGMELAELYARLVDDREIADRILGVISAEFDLTRQMFGVVTGSEDLLADNPALARSVRRRFPYLLPLNIIQLEMLRRHRAGDDREVVSRGIQLTMNGLATALRNSG

SEQ ID NO: 7 - protein, 923 aa, Corynebacterium humireducens
MTVRDHLQEDIRYLGRILGQVIAEQEGEDVFNLVEHARQQAFEVAKGNASLEVLVDLFRNIEPERATPVIRAFSHFALMANLAEDIHDDFHRERILDEGGAPDSTLNATWEKFREAGVSAADIERSLSAGLVAPVLTAHPTETRRRTVFDAQKHITELMLQRHAVQDAEPNARTEDRLAEIERNIRRRMTILWQTALIRQARPRIEDEIEVGLRYYTLSLLREIPALNRHVVDTLTERFGADLRNSAGQAIVRPGSWIGGDHDGNPFVTADTLDYASRRAAQTVLKYYVTQLHALEHELSLSDRMTSVTVELVALAGRGKNDVPSRVDEPYRRAVHGVRGRILATTAALIGEDAVEGVWHREHEPYGSPQEFEADLRVIDTSLRASHDEIIADDRLSSIRAAVASFGFHLYSIDLRQNSESFENVLTEVFATAHVHPNYDTLREEVKIELLTRELQTPRPLVPRGYRGFSEPTQRELDLISQAADSVARFGEQMIPHQIISMAQSVSDILEPMVLLKEVGLIQANGQGPTGSVDIIPLFETIDDLQAGAGILRELWDLPIYRAYLAQRGDIQEVMLGYSDSNKDGGYFAANWALYDAETDLVKVGREYGVRLRLFHGRGGTVGRGGGPSYDALLAQPQGAVDGSVRITEQGEIISAKYGSPRTARRNLEALVSATLEASLLTVDDLADRARATQIMSELAQISRRKYSELVHEDPGFIPYFTQSTPLDEIGSLNIGSRPTARKQTKGVDDLRAIPWVLAWSQSRVLLPGWFGVGTALAEWIGEGEDRDERIAELRELYESWPFFTSIMSNMAQVMSKAGMDLAELYARLIDDREVAERVHGVITEEFELTREMFSTVTGSAELLADNPALARSVRRRFPYLLPLNIIQLELLRRHRAGDSRRAVSRGIQLTMNGLATALRNSG

SEQ ID NO: 8 - protein, 921 aa, Corynebacterium halotolerans
MTPADHLQEDIRYLGRVLGRVIAEQEGEEVFDLVEHARQLAFGIAKGDTGLEALVELFRNIEPARATPVIRAFSHFALMANLAEDLHDDFTRERLLDAGGPAPDSTLETTWAKLAGAGVDAATVAEALDGALVAPVLTAHPTETRRRTVFDAQRHITEQMTVRHRLLEAPHTARTDSQLEEVDRQIRRRLTILWQTALIRMARPRIEDEIEVGLRYYKLSLLEEIPALNRAVDDRLRADFGQGVPTRALVRPGSWIGGDHDGNPFVTAATLDYATRRAAQTVLKHYETQLLALEHELSLSDRMTSVTVDLVALAKRGLNDVPSRVDEPYRRAIRGIRGRLLATTATLIGEDAVEGSWHRVHEPYSGPGEFDADLAVIDESLRRSHDEIIADDRLATIRAAVAGFGFHLYSLDLRQNSESFEAILAEVFAAAGVHDDYASLGEEERIDLLTRELRTPRPLVPRGRRGFSEPTQRELDLFEQAATSVERFGMEMIPHLIISMATSVSDILEPMVLLKEVGLLHADGERPTGSVDVIPLFETIDDLAAGAGILRELWALPLYRAYLAQRGDVQEVMLGYSDSNKDGGYLAANWALYDAETQIVAAGRDHGVRLRLFHGRGGTVGRGGGPSYEAILAQPKGAVDGSVRITEQGEIISAKYGSGHTARRNLEALVSATLEASLLDVDELVDRERATGIMTEIARLSRARYSRLVHEDPGFIPYFTQSTPLDEIGALNIGSRPTSRKQTNSVADLRAIPWVLAWSQSRVMLPGWFGVGTALAEWIGEGDDAEDRCAELRHLYETWPFFTSVMSNMAQVMSKAGMELAGLYAGLVDDREVAGRIRGIISEEFRLTREMFTKVTGSEDLLADNPMLARSVRRRFPYLLPLNIIQVELLRRHRAGDERDAVSRGIRLTMNGLATALRNSG

SEQ ID NO: 9 - protein, 927 aa, Corynebacterium deserti
MEKSVYKVMTDFLRDDIRFLGRILGEVIAEQEGREVYELVEKARQISFEIAKGNADMDSLVTVFDGISPAEATPIARAFTHFALLANLAEDLHDEQTREKALDAGETPPDSTLDATWLKLNDANTDAQAVTEFMNNAQVAPVLTAHPTETRRRTVFDAQKWITTHMRERHLIQTSTETARTQAKLDEIERSIRRRITILWQTALIRVARPRIEDEIEVGLRYYKLSLLEEIPKINRDVNLELRQRFGEDIPNKAVIKPGSWIGGDHDGNPYVTADTVEYSTHRAAQTVLKYYTRQLHSLEHELSLSDRMNAVTQELSKLADAGNNDVPSRVDEPYRRAVHGVRGRILATTAHLIGEDAVEGVWFRQFEPYTSPEEFLADLVTVDQSLRASNDDLIADDRLAKLISAVESFGFNLYSLDLRQNSESYEDVLTELFQRAVVTDNYRDMSEEEKLELLLAELRSPRPLIPHGAEGYSEPTDRELGIFRKASEAVQKFGPRMVPHCIISMASSVTDVLEPMVLLKEFGLIAANGDSPTGTVDVIPLFETIEDLQAGSGILEELWGIDLYRNYLEQRGMTQEVMLGYSDSNKDGGYFAANWALYDAELHLVELCRAAGVKLRLFHGRGGTVGRGGGPSYDAILAQPKGAVLGSVRITEQGEIISAKYGNPETARRNLEALVSATLEATLLDVSDLADPERAYTIMREISELSLKKYSSLVHEDPGFISYFTQSTPLREIGSLNIGSRPSSRKQTSSVDDLRAIPWVLSWSQSRVMLPGWFGVGSALEEWIGSGEEAEARIAELQTLNESWPFFTSVLDNMAQVMSKAELRLAKLYADLIPDQEVAERIYTDIFEEYFLTKKMFCKITGSSDLLDDNPLLARSVQRRYPYLLPLNVIQVEMMRRFRSGDDSDGISRNIQLTMNGLSTALRNSG

SEQ ID NO: 10 - protein, 914 aa, Corynebacterium doosanense
MTEQVRDDIRFLGRILGRVIAEQEGEDVFELVESTRQLAFGVARGDEDAEALLATFRGVDENKINLVARSFSHFSLMANIAEDLDDEAALAALEDEGSQAPDASLQGALAKLRADGTVSPGDVATMLDNAQVSPVFTAHPTETRRRTVFDVQSRIVALLRERRGILAQPRTPRRDARLTQIEREAHLRMTLLWQTALIRIARPRIEDEVNVGLRYFRLSLLDEVPAINRDTIAGLRELFGAGVPDRPLVRTGSWIGGDHDGNPFVTGETLTYATQAAADTVLDYYDEQLGELEKELSLSDRYSECSQELRELADRGNNDVPSRVDEPYRRALHGVLGRIRATRCALADAPDSDGFDPYPTPQDFSADLDIIDRSLRQFDDAIIADDRLLRIRSAAETFGFHLNSLDLRQNSESFEAVLGELFAAAGVTADYAGLDEAAKCELLVAELTSARPLTFPWAEPFSEPTERELGIFRAAAGAVDKLGPEVIPHCIVSMTGTVSDILEPMILLKEFGLISFDPERGQLVGQVDIAPLFETIDDLKAGARILEELWDVPVYRQYLRQRDDLQEVVLGYSDSNKDGGYLSANWELYDAQIAIVEACRNHDIRLRFSHGRGGAVGRGGGPTYDAILAQPVGAVRGSVRITEQGEVISAHYGTATTARRHLEAFVAGTLEASLLDTESLDNPTRAYEIMREIAELAGGKYGDLIRRDPGFIDYFTQSTPLHEIGDLNLGSRPTARKQTSSVSDLRAIPWVLSWAQSRVNLPGWFGVGTAITCWAGDDETRWEELRTLYRTWSFFRSVMDNMAQVMGKASMDLARIYSTLVDDPEVSERVFSTIADEFELTQSVFHRITGHESLMAGNDRLERSVQRRYPYLLPLNAIQIELLRRYRSGDDSFLVSKTIQVTMNGLATGLRVSG

SEQ ID NO: 11 - protein, 923 aa, Corynebacterium pollutisoli
MTVRDHLQEDIRYLGRILGQVIAEQEGEDVFNLVEHARQQAFEVAKGNASLEVLVDLFRNIDPERATPVVRAFSHFALMANLAEDIHDDFNRERILDEGGAPDSTLEATWEKFDEADVSAGDIETSLSAALVAPVLTAHPTETRRRTVFDAQKHITDLMLARHAILDAEETARTEARLADVERNIRRRMTILWQTALIRQARPRIEDEIEVGLRYYTLSLLKEIPALNRHVHDSLTDRFGADLQGSNGQAIVRPGSWIGGDHDGNPFVTAATLDYASRRAAQTVLKYYAQQLHALEHELSLSDRMTSVTVELVALAGKGRNDVPSRVDEPYRRAVHGVRGRILATTAHLIGEDAVEGTWHREHEPYIDPSEFDADLKVIDTSLRASQDEIIADDRLSTIRAAIASFGFHLYSIDLRQNSESFENVLTEVFATAHVHPNYDTLREEEKVELLVRELQTPRPLVPRGYRGFSEATQRELDLITQAAVSVERFGEQMIPHQIISMAQSVSDILEPMVLLKEVGLIRANGEGPTGSVDIIPLFETIDDLQAGAGILRKLWDLPIYRAYLRQRGDIQEVMLGYSDSNKDGGYFAANWALYDAETDLVEVGREYGVRLRLFHGRGGTVGRGGGPSYDAILAQPQGAVDGSVRITEQGEIISAKYGSERAARRNLEALVSATLEASLLTVDDLEDRARATRIMSELSAISRRKYSELVHEDPGFIPYFTQSTPLHEIGSLNIGSRPTSRKQTKGVEDLRAIPWVLAWSQSRVLLPGWFGVGTALDEWIGDGEDREERIAELRHLYETWPFFASIMSNMAQVMSKAGMDLAELYARLIDDREVADRVHGVITAEFELTRGMFSTVTGSEELLADNPALARSVRRRFPYLLPLNIIQLEMLRRHRAGDARQAVSRGIQLTMNGLATALRNSG

SEQ ID NO: 12 - protein, 940 aa, Arthrobacter sp.
MAHTAMNPETDLASELRADVRRVSTLLGESLVRQHGPELLDLVEQVRLLTKESKEAARGGADATGPWSAHDVVAQVRELLGSLPIGQATDLVRAFAFYFHLANAAEQVHRVRGLRTRAEKDGWLAKTIADIASQAGPGVLQEVVNGLDVRPIFTAHPTEASRRSVLDKIRKLSDVLAQPTAEGTTARRRQDRQLAEIIDQMWQTDELRQVRPTPVDEARNAIYYLGSILTDAMPEMLTEFSDLLSEHGVTLASQDAPIRFGSWIGGDRDGNPNVTAAVTREILQIQNQHAVRISIGMIDELISILSNSTALAGADQELLDSIDSDLKNLPGLDKRVLELNAQEPYRLKLTCIKAKLINTGKRVAAGSNHEHGRDYSGTDELLADLELLERSLRNHSASLAADGALARVRRAIASFGLHLATLDIREHADHHHDAVGQLMDRLGGPGLRYAELSREERFEVLGSELASRRPLSGHPIKLDGAADGTYDVFREIRRALRTYGPDVIETYIISMTRGADDVLAAAVLAREAGLVNLFGEKPYAKLGFAPLLETVEELRASAEIVDQLLSDPSYRELVRLRGDVQEVMLGYSDSNKESGVMTSQWEIHKTQRKLRDVAAKHGVRVRLFHGRGGSVGRGGGPTYDAILAQPNGVLEGEIKFTEQGEVISDKYSLPELARENLELSLAAVLQGSALHKDPRTSADQRERYGHVMETISDAAFDRYRNLIDNPDLPAYFMASTPVEQLGSLNIGSRPSKRPDSGAGLGGLRAIPWVFGWTQSRQIVPGWFGVGSGLKAAREAGNAAQLVEMWENWHFFRSVLSNVEMTLAKTDLDIAGYYVSTLVPEELHHIFRSIREEYELTVAEVQNLTGESLLLDAQPTLKRSLEIRDQYLDPISYLQVELLRRVRAEAADAADGISGAEIDERLQRAMLITVNGVAAGLRNTG

SEQ ID NO: 13 - protein, 883 aa, Escherichia coli
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 14 - DNA, 33 bp, synthetic construct
  source 1..33: mol_type = other DNA
acgacggccagtgaacgttgtgtgcccatgcaa

SEQ ID NO: 15 - DNA, 34 bp, synthetic construct
  source 1..34: mol_type = other DNA
acatatttgttcggaagccaattcccccacaatc

SEQ ID NO: 16 - DNA, 22 bp, synthetic construct
  source 1..22: mol_type = other DNA
tccgaacaaatatgtttgaaag

SEQ ID NO: 17 - DNA, 34 bp, synthetic construct
  source 1..34: mol_type = other DNA
ggtaccgagctcgaattgacgattgctgcacgtg

(19) United States
(12) Patent Application Publication
(10) Pub. No.: US 2025/0197901 A1
(43) Pub. Date: Jun. 19, 2025

(54) GENETICALLY MODIFIED MICROORGANISM AND METHOD FOR PRODUCING ASPARTIC ACID

(71) Applicants: DIC Corporation, Tokyo (JP); GREEN EARTH INSTITUTE CO., LTD., Tokyo (JP)

(72) Inventors: Yuji Ishigaki, Sakura-shi (JP); Makoto Sugimoto, Sakura-shi (JP); Toru Nakayashiki, Kisarazu-shi (JP); Yakufu Aimaier, Kisarazu-shi (JP)

(73) Assignees: DIC Corporation, Tokyo (JP); GREEN EARTH INSTITUTE CO., LTD., Tokyo (JP)

(21) Appl. No.: 18/712,730

(22) PCT Filed: Nov. 25, 2022
(86) PCT No.: PCT/JP2022/043626

§ 371 Date: Dec. 10, 2024

(30) Foreign Application Priority Data
Nov. 26, 2021 (JP) 2021-192344

(51) Int. Cl. C12P 13/20; C12N 1/20; C12N 9/02; C12N 9/04; C12N 9/10; C12N 9/88; C12R 1/15

(52) U.S. Cl. CPC C12P 13/20; C12N 1/20; C12N 9/0006; C12N 9/0008; C12N 9/001; C12N 9/1025; C12N 9/88; C12N 2510/00; C12R 2001/15; C12Y 101/01027; C12Y 102/05001; C12Y 103/05001; C12Y 203/03001; C12Y 401/01031

(57) ABSTRACT

A genetically modified microorganism which satisfies at least one condition selected from the group consisting of the following conditions (I) and (II). Condition (I): citrate synthase activity is reduced or inactivated compared with a wild-type microorganism corresponding to the genetically modified microorganism, and condition (II): oxaloacetate decarboxylase activity is reduced or inactivated compared with the wild-type microorganism.

### TECHNICAL FIELD

The present invention relates to a genetically modified microorganism and a method for producing aspartic acid.

Priority is claimed on Japanese Patent Application No. 2021-192344, filed Nov. 26, 2021, the content of which is incorporated herein by reference.

### SEQUENCE LISTING

The instant application contains a Sequence Listing which has been submitted in XML format and is hereby incorporated by reference in its entirety. The XML Sequence Listing file is named 1420149.1042US9-Sequence Listing.xml, was created on Dec. 4, 2024 and is 30,069 bytes (28.2 KB) in size.

### BACKGROUND ART

In recent years, in order to improve environmental awareness, attempts have been made to use a bio-based raw material instead of a petroleum-based raw material. In the bio-based raw material, the yield tends to be low and the cost tends to be high compared with the petroleum-based raw material. Therefore, there is a demand for a technique of producing a bio-based raw material at a low cost and a high yield by using microorganisms or the like.

Aspartic acid is polymerized to be used as a polymeric absorbing polymer for paper diapers and a thickener for cosmetics. The production of aspartic acid by a biotechnological method is mainly performed by a bioreactor using immobilized bacterial cells of Escherichia coli having aspartase activity, from fumaric acid as a raw material. There are few reports on a technique for producing aspartic acid from glucose, which is cheaper than fumaric acid.

For example, in Non-Patent Document 1, it is reported that by deleting pyruvate kinase (PYK), the production amount of glutamic acid from glucose is increased and aspartic acid is produced.

### CITATION LIST

### Non-Patent Document

Non-Patent Document 1
Kazunori Sawada, Susumu Zen-in, Masaru Wada, Atsushi Yokota, Metabolic changes in a pyruvate kinase gene deletion mutant of Corynebacterium glutamicum ATCC 13032. Metabolic Engineering 12 (2010) 401-407.

### SUMMARY OF INVENTION

### Technical Problem

In order to produce aspartic acid at a low cost by microbial fermentation, it is required to improve the yield of aspartic acid. In addition, in order to reduce the purification cost of aspartic acid, it is required to reduce the production amount of by-products such as other amino acids and organic acids.

Therefore, an object of the present invention is to provide a genetically modified microorganism and a method for producing aspartic acid or a derivative thereof, in which a production amount and a yield of aspartic acid or a derivative thereof can be improved and a production amount of by-products (such as amino acids other than aspartic acid, and organic acids) can be reduced.

### Solution to Problem

The present invention includes the following aspects.
[1] A genetically modified microorganism that satisfies at least one condition selected from the group consisting of the following conditions (I) and (II),
condition (I): citrate synthase activity is reduced or inactivated compared with a wild-type microorganism corresponding to the genetically modified microorganism, and
condition (II): oxaloacetate decarboxylase activity is reduced or inactivated compared with the wild-type microorganism.
[2] The genetically modified microorganism according to [1], in which the genetically modified microorganism further satisfies at least one condition selected from the group consisting of the following conditions (III) to (VI),
condition (III): succinate dehydrogenase activity or fumarate reductase activity is reduced or inactivated compared with the wild-type microorganism,
condition (IV): lactate dehydrogenase activity is reduced or inactivated compared with the wild-type microorganism,
condition (V): modified phosphoenolpyruvate carboxylase activity that exhibits resistance to feedback inhibition by aspartic acid in wild-type phosphoenolpyruvate carboxylase activity, or exogenous phosphoenolpyruvate carboxylase activity that exhibits higher resistance to feedback inhibition by aspartic acid compared with the wild-type phosphoenolpyruvate carboxylase activity exhibited by the wild-type microorganism is provided, and
condition (VI): pyruvate: quinone oxidoreductase activity is reduced or inactivated compared with the wild-type microorganism.
[3] The genetically modified microorganism according to [1] or [2], in which the genetically modified microorganism satisfies the condition (I).
[4] The genetically modified microorganism according to any one of [1] to [3], in which the genetically modified microorganism belongs to a Gram-positive bacterium.
[5] The genetically modified microorganism according to any one of [1] to [4], in which the genetically modified microorganism is used for producing aspartic acid or a derivative of aspartic acid.
[6] The genetically modified microorganism according to [5], in which a production amount of at least one by-product selected from the group consisting of amino acids other than the aspartic acid and organic acids is reduced compared with a microorganism not satisfying the condition (I).
[7] The method for producing aspartic acid or a derivative of aspartic acid, the method including (p) producing aspartic acid or a derivative of aspartic acid using a cell of the genetically modified microorganism according to any one of [1] to [6] or a treated product of the cell and (q) recovering the aspartic acid or the derivative of aspartic acid.
[8] The method for producing aspartic acid or a derivative of aspartic acid according to [7], in which the (p) is performed in a reaction medium having a dissolved oxygen concentration of 0.5 mg/L or less.
[9] The method for producing aspartic acid or a derivative of aspartic acid according to [7] or [8], in which a production amount of at least one by-product selected from the group consisting of amino acids other than the aspartic acid and organic acids is reduced compared with a case of producing the aspartic acid using a cell of a microorganism not satisfying the condition (I) or a treated product of the cell.

### Advantageous Effects of Invention

According to the present invention, a genetically modified microorganism and a method for producing aspartic acid or a derivative thereof, in which a production amount and a yield of aspartic acid or a derivative thereof can be improved and a production amount of by-products (such as amino acids other than aspartic acid, and organic acids) can be reduced, are provided.

### BRIEF DESCRIPTION OF DRAWINGS

FIG. 1 A schematic diagram showing an example of a metabolic pathway involved in the production of aspartic acid. Glc: glucose, PEP: phosphoenolpyruvic acid, Pyr: pyruvic acid, Ac-COA: acetyl COA, Ac: acetic acid, Cit: citric acid, α-keto: α-ketoglutaric acid, Fum: fumaric acid, Mal: L-malic acid, OAA: oxaloacetic acid, Asp: aspartic acid, Ldh: lactate dehydrogenase, PoxB: pyruvate: quinone oxidoreductase, Odx: oxaloacetate decarboxylase, PlgltA: promoter P1 of citrate synthase gene, Sdh: succinate dehydrogenase, AspA: aspartate ammonia lyase, and mutant Ppc: phosphoenolpyruvate carboxylase.

FIG. 2 A schematic diagram showing a preparation method for a PlgltA-deficient strain prepared in Examples.

### DESCRIPTION OF EMBODIMENTS

### <Genetically Modified Microorganism>

A first aspect of the present invention is a genetically modified microorganism that satisfies at least one condition selected from the group consisting of the following conditions (I) and (II),
condition (I): citrate synthase activity is reduced or inactivated compared with a wild-type microorganism corresponding to the genetically modified microorganism, and
condition (II): oxaloacetate decarboxylase activity is reduced or inactivated compared with the wild-type microorganism.

In one embodiment, the genetically modified microorganism satisfies at least one condition selected from the group consisting of the following conditions (III) to (VI) in addition to the above-described conditions (I) and/or (II),
condition (III): succinate dehydrogenase activity or fumarate reductase activity is reduced or inactivated compared with the wild-type microorganism,
condition (IV): lactate dehydrogenase activity is reduced or inactivated compared with the wild-type microorganism,
condition (V): modified phosphoenolpyruvate carboxylase activity that exhibits resistance to feedback inhibition by aspartic acid in wild-type phosphoenolpyruvate carboxylase activity, or exogenous phosphoenolpyruvate carboxylase activity that exhibits higher resistance to feedback inhibition by aspartic acid compared with the wild-type phosphoenolpyruvate carboxylase activity exhibited by the wild-type microorganism is provided, and condition (VI): pyruvate: quinone oxidoreductase activity is reduced or inactivated compared with the wild-type microorganism.

In one embodiment, the genetically modified microorganism satisfies the above-described condition (I).

In one embodiment, the genetically modified microorganism satisfies the above-described condition (II).

In one embodiment, the genetically modified microorganism satisfies the above-described conditions (I) and (II).

The “genetically modified microorganism” can be understood literally and may be understood to be a microorganism on which any genetic modification manipulation has been performed. More specifically, such a genetic modification manipulation may realize the condition (I) or (II) or the conditions (I) to (VI) in any combination within the range defined for the genetically modified microorganism according to the first aspect.

The term “microorganism” can be interpreted literally, and more specifically, the term “microorganism” may be a prokaryote such as a bacterium, an archaeon, or a cyanobacterium, or a eukaryote such as a fungus. The “microorganism” is preferably a fungus or bacteria and more preferably bacteria.

Examples of the fungi include the genus Saccharomyces (for example, Saccharomyces cerevisiae), the genus Schizosaccharomyces (for example, Schizosaccharomyces pombe), the genus Pichia (for example, Pichia pastoris), the genus Kluyveromyces (Kluyveromyces lactis), Hansenula polymorpha, the genus Yarrowia (for example, Yarrowia lipolytica), the genus Cryptococcus (for example, Cryptococcus sp. S-2), the genus Aspergillus (for example, Aspergillus oryzae), and the genus Pseudozyma (for example, Pseudozyma antarctica). Saccharomyces cerevisiae, Schizosaccharomyces pombe, Pichia pastoris, and the like can be suitably used, for which genetic modification techniques or heterologous protein expression systems have been established.

Examples of the bacteria include the genus Escherichia (for example, Escherichia coli), the genus Bacillus (for example, Bacillus subtilis), the genus Lactobacillus (for example, Lactobacillus acidophilus), the genus Clostridium (for example, Clostridium thermocellum and Clostridium acetobutylicum), the genus Rhodopseudomonas (for example, Rhodopseudomonas palustris), the genus Rhodobacter (Rhodobacter capsulatus), the coryneform bacteria which will be described below, and the like. As the bacteria, since genetic modification techniques and a protein expression system have already been established, the bacterium of the genus Escherichia or the coryneform bacterium is preferable, an Escherichia coli or the coryneform bacterium is more preferable, and the Corynebacterium is still more preferable.

In some embodiments, the genetically modified microorganism according to the present aspect is a Gram-positive bacterium (for example, an actinomycete). In some other embodiments, the genetically modified microorganism according to the present aspect may be a Gram-negative bacterium. Examples of the Gram-negative bacterium include a bacterium belonging to the phylum Proteobacteria. The bacterium of the phylum Proteobacteria include a bacterium belonging to the class alpha-, beta-, gamma-, delta-, epsilon-, or zeta-Proteobacteria, and a bacterium belonging to the class Oligoflexus. Examples of the Gram-negative bacterium include a bacterium belonging to the Enterobacteriaceae, the Vibrionaceae, or the Pseudomonadaceae.

The “coryneform bacterium” refers to a group of bacteria defined in Bergey's Manual of Determinative Bacteriology (Vol. 8, p. 599, 1974).

Examples of the coryneform bacterium include the genus Corynebacterium, the genus Brevibacterium, the genus Arthrobacter, the genus Mycobacterium, the genus Micrococcus, the genus Microbacterium, and the like.

Examples of the genus Corynebacterium include the following species and bacterial strains.

Corynebacterium glutamicum (for example, FERM P-18976 strain, ATCC13032 strain, ATCC31831 strain, ATCC13058 strain, ATCC13059 strain, ATCC13060 strain, ATCC13232 strain, ATCC13286 strain, ATCC13287 strain, ATCC13655 strain, ATCC13745 strain, ATCC13746 strain, ATCC13761 strain, and ATCC14020 strain); Corynebacterium acetoglutamicum (for example, ATCC15806 strain);
Corynebacterium acetoacidophilum (for example, ATCC13870 strain);
Corynebacterium melassecola (for example, ATCC17965 strain);
Corynebacterium efficiens (for example, YS-314 strain, and YS-314 strain (NBRC100395 strain));
Corynebacterium alkanolyticum (for example, ATCC21511 strain);
Corynebacterium callunae (for example, ATCC15991 strain, NBRC15359 strain, and DSM20147 strain);
Corynebacterium lilium (for example, ATCC15990 strain);
Corynebacterium thermoaminogenes (Corynebacterium efficiens)) (for example, AJ12340 strain, and FERM BP1539 strain);
Corynebacterium herculis (for example, ATCC13868 strain);
Corynebacterium ammoniagenes (Corynebacterium stationis) (for example, ATCC6871 strain, ATCC6872 strain, DSM20306 strain, NBRC12071 strain, NBRC12072 strain, and NBRC12612 strain);
Corynebacterium pollutisoli;
Corynebacterium marinum (for example, DSM44953 strain);
Corynebacterium humireducens (for example, NBRC106098 strain);
Corynebacterium halotolerans (for example, YIM70093 strain);
Corynebacterium deserti (for example, GIMN1.010 strain);
Corynebacterium doosanense (for example, CAU212 strain, and DSM45436 strain); and
Corynebacterium maris (for example, DSM45190 strain).

Examples of the bacteria of the genus Brevibacterium include the following species and bacterial strains.
Brevibacterium divaricatum (for example, ATCC14020 strain);
Brevibacterium flavum [for example, strain MJ-233 (FERM BP-1497) strain, MJ-233AB-41 (FERM BP-1498) strain, ATCC13826 strain, ATCC14067 strain, and ATCC13826 strain];
Brevibacterium immariophilum (for example, ATCC14068 strain);
Brevibacterium lactofermentum (Corynebacterium glutamicum) (for example, ATCC13869 strain);
Brevibacterium roseum (for example, ATCC13825 strain);
Brevibacterium saccharolyticum (for example, ATCC14066 strain);
Brevibacterium thiogenitalis (for example, ATCC19240 strain);
Brevibacterium album (for example, ATCC15111 strain);
Brevibacterium cerinum (for example, ATCC15112 strain).

Examples of the bacteria of the genus Arthrobacter include the following species and bacterial strains.
Arthrobacter globiformis (for example, ATCC8010 strain, ATCC4336 strain, ATCC21056 strain, ATCC31250 strain, ATCC31738 strain, ATCC35698 strain, NBRC3062 strain, and NBRC12137T strain) and the like are included.

Examples of the bacteria of the genus Micrococcus include Micrococcus freudenreichii [for example, No. 239 (FERM P-13221) strain]; Micrococcus luteus [for example, NCTC2665 strain, strain No. 240 (FERM P-13222) strain]; Micrococcus ureae (for example, IAM1010 strain); Micrococcus roseus (for example, IFO3764 strain); and the like.

Examples of the bacteria of the genus Microbacterium include Microbacterium ammoniaphilum (for example, ATCC15354 strain) and the like.

The coryneform bacterium strain can be supplied by, for example, in a case of ATCC strain, the American Type Culture Collection (P. O. Box 1549 Manassas, VA 20108 USA). Other bacterial strains can also be supplied by respective microbial culture collections that provide the bacterial strains.

The genetically modified microorganism according to the present aspect can be prepared by subjecting the microorganism exemplified above to a predetermined genetic manipulation.

### (Regarding Conditions (I) to (IV) and (VI))

The expression “the citrate synthase activity is reduced or inactivated compared with the wild-type microorganism corresponding to the genetically modified microorganism” in condition (I) means that the citrate synthase activity is significantly reduced or completely inactivated compared with the wild-type microorganism. The “wild-type microorganism” means a microorganism on which no genetic manipulation has been performed. The wild-type microorganism may be a microorganism isolated from nature, or may be an established microorganism strain. The “wild-type microorganism corresponding to the genetically modified microorganism” means a wild-type microorganism having the same genetic background as the genetically modified microorganism. The genetically modified microorganism of the present aspect may be obtained by subjecting a corresponding wild-type microorganism to a genetic manipulation to realize the condition (I) and/or the condition (II), or any one or more of the conditions (III) to (VI) in addition to the condition (I) and/or the condition (II). In a case where the citrate synthase activity is reduced compared with the wild-type microorganism, the citrate synthase activity of the genetically modified microorganism may be, for example, 90 or less, 80 or less, 70 or less, 60 or less, or 50 or less as the relative activity in a case where the citrate synthase activity of the wild-type microorganism is set to 100. The same applies to the conditions (II) to (IV) and (VI).

The expression “the oxaloacetate decarboxylase activity is reduced or inactivated compared with the wild-type microorganism” in the condition (II) means that the oxaloacetate decarboxylase activity is significantly reduced or completely inactivated compared with the wild-type microorganism.

The expression “the succinate dehydrogenase activity or fumarate reductase activity is reduced or inactivated compared with the wild-type microorganism corresponding to the genetically modified microorganism” in the condition (III) means that the succinate dehydrogenase activity or fumarate reductase activity is significantly reduced or completely inactivated compared with the wild-type microorganism. Some bacteria such as the genus Corynebacterium do not have fumarate reductase, and succinate dehydrogenase catalyzes this reaction. Some bacteria such as Escherichia coli have both the succinate dehydrogenase and the fumarate reductase, and mainly the fumarate reductase catalyzes the reaction.

The expression “the lactic acid dehydrogenase activity is reduced or inactivated compared with the wild-type microorganism” in the condition (IV) means that the lactic acid dehydrogenase activity is significantly reduced or completely inactivated compared with the wild-type microorganism.

The expression “the pyruvate: quinone oxidoreductase activity is reduced or inactivated compared with the wild-type microorganism” in the condition (VI) means that the pyruvate: quinone oxidoreductase activity is significantly reduced or completely inactivated compared with the wild-type microorganism.

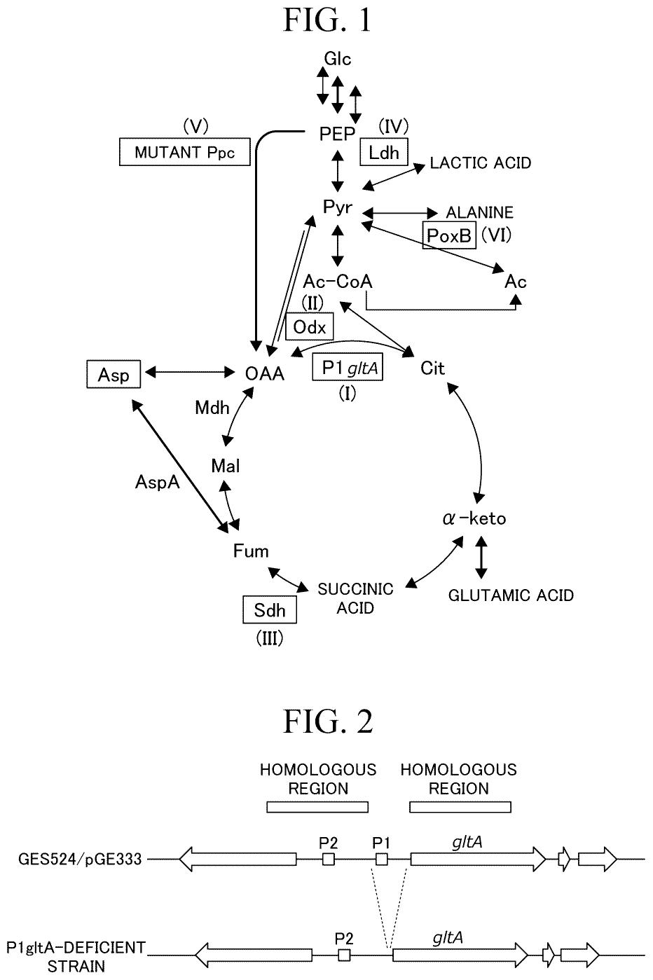

As shown in FIG. 1, the meanings of each of the conditions (I) to (IV), and (VI) are that, in the metabolic pathway of the microorganism, the metabolism from oxaloacetic acid (OAA) to citric acid (Cit), the metabolism from oxaloacetic acid (OAA) to pyruvic acid (Pyr), the metabolism from succinic acid to fumaric acid (Fum) or the reverse metabolism thereof, the metabolism from pyruvic acid (Pyr) to lactic acid, and the metabolism from pyruvic acid (Pyr) to acetic acid (Ac), respectively, are significantly suppressed or inactivated.

In the microorganism that can proliferate under aerobic conditions and does not proliferate under reducing conditions (anaerobic conditions), generally, in the TCA cycle (citric acid cycle) shown in FIG. 1, metabolism proceeds clockwise from oxaloacetic acid under aerobic conditions, and on the other hand, metabolism proceeds counterclockwise from oxaloacetic acid under reducing conditions or anaerobic conditions.

In a case of adopting the embodiment in which the condition (I) is satisfied, since the conversion from oxaloacetic acid to citric acid is suppressed under the aerobic conditions, a larger amount of oxaloacetic acid is accumulated. As a result, production of further metabolites derived from oxaloacetic acid can be efficiently performed. On the other hand, under the reducing conditions or the anaerobic conditions, a larger amount of oxaloacetic acid, L-malic acid, fumaric acid, or further metabolites derived therefrom can be efficiently produced. In the genetically modified microorganism satisfying the condition (I), the further metabolites derived from the metabolite in the TCA cycle may be biosynthesized through a metabolic system retained by a corresponding wild-type microorganism or may be biosynthesized through a metabolic system newly constructed by further introducing any gene mutation.

In a case of adopting the embodiment in which the condition (II) is satisfied, since the conversion from oxaloacetic acid to pyruvic acid is suppressed, a larger amount of oxaloacetic acid is accumulated in the same manner as in the embodiment satisfying the condition (I). As a result, production of further metabolites derived from oxaloacetic acid can be efficiently performed.

In a case of adopting the embodiment in which the condition (III) is satisfied, since the conversion from succinic acid to fumaric acid is suppressed under the aerobic conditions, a larger amount of citric acid, cis-aconitic acid, D-isocitric acid, α-ketoglutaric acid, succinyl CoA, succinic acid, or further metabolites derived therefrom can be efficiently produced. On the other hand, under the reducing conditions or the anaerobic conditions, a larger amount of oxaloacetic acid, L-malic acid, fumaric acid, or further metabolites derived therefrom can be efficiently produced.

In a case of adopting the embodiment in which the condition (IV) is satisfied, since the conversion from pyruvic acid to lactic acid is suppressed, the metabolic pathway from pyruvic acid to oxaloacetic acid proceeds efficiently. As a result, production of oxaloacetic acid, L-malic acid, fumaric acid, or further metabolites derived therefrom can be efficiently performed.

In a case of adopting the embodiment the condition (VI) is satisfied, since the conversion from pyruvic acid to acetic acid is suppressed, the metabolic pathway from pyruvic acid to oxaloacetic acid proceeds efficiently, similarly to the embodiment the condition (IV) is satisfied. As a result, production of oxaloacetic acid, L-malic acid, fumaric acid, or further metabolites derived therefrom can be efficiently performed.

The embodiment in which both the conditions (IV) and (VI) are satisfied is preferable since the production efficiency of oxaloacetic acid, L-malic acid, fumaric acid, or further metabolites derived therefrom can be further improved.

In one embodiment, the genetically modified microorganism satisfies the condition (I).

In one embodiment, the genetically modified microorganism satisfies the condition (I) and further satisfies at least one condition selected from the group consisting of the conditions (II) to (IV) and (VI). In this case, the genetically modified microorganism preferably satisfies both of the conditions (I) and (II), both of the conditions (I) and (III), both of the conditions (I) and (IV), both of the conditions (I) and (VI), or the conditions (I), (II), and (III), more preferably satisfies the conditions (I), (II), (III), and (VI), and more preferably satisfies all of the conditions (I), (II), (III), (IV), and (VI).

In one embodiment, the genetically modified microorganism satisfies the condition (II).

In one embodiment, the genetically modified microorganism satisfies the condition (II) and further satisfies at least one condition selected from the group consisting of the conditions (I), (III), (IV), and (VI). In this case, the genetically modified microorganism preferably satisfies both of the conditions (I) and (II), both of the conditions (II) and (III), both of the conditions (II) and (IV), both of the conditions (II) and (VI), or the conditions (I), (II), and (IV), more preferably satisfies the conditions (I), (II), (IV), and (VI), and more preferably satisfies all of the conditions (I), (II), (III), (IV), and (VI).

In such an embodiment, this is because the metabolic pathway from pyruvic acid to the TCA cycle and the metabolism in the TCA cycle proceed efficiently, and it is possible to achieve efficiently the production of oxaloacetic acid, L-malic acid, fumaric acid, or metabolite derived therefrom, and substances derived from these metabolites or further metabolism from these metabolites in the TCA cycle.

The genetically modified microorganism may further satisfy the following condition (VII) in addition to the above-described conditions.

Condition (VII): pyruvate formate lyase activity is reduced or inactivated compared with the wild-type microorganism.

The expression “pyruvate formate lyase activity is reduced or inactivated compared with the wild-type microorganism” in the condition (VII) means that the pyruvate formate lyase activity is significantly reduced or completely inactivated compared with the wild-type microorganism.

In a case where the genetically modified microorganism is a Gram-negative bacterium, it is preferable to satisfy the condition (VII). The Gram-negative bacterium exhibits pyruvate formate lyase activity that is not usually observed in the Gram-positive bacterium. The pyruvate formate lyase activity creates a secondary biosynthetic pathway that synthesizes an organic acid such as formic acid and acetic acid from pyruvic acid. That is, since the secondary biosynthetic pathway can be blocked in a case where pyruvate formate lyase activity is reduced or inactivated, the metabolic flux to aspartic acid is made more stable, and aspartic acid can be efficiently produced.

The citrate synthase activity according to the condition (I), the oxaloacetate decarboxylase activity according to the condition (II), the succinate dehydrogenase activity or fumarate reductase activity according to the condition (III), the lactate dehydrogenase activity according to the condition (IV), the pyruvate: quinone oxidoreductase activity according to the condition (VI), and the pyruvate formate lyase activity according to the condition (VII) may be each enzyme activity of each enzyme shown in each of the conditions. More specifically, the enzyme can be described by an EC number that is recognized as a systematic classification according to the type of reaction between a substrate and an enzyme and an international enzyme classification based on the type of reaction species. Examples of the enzyme carrying the enzyme activity under each condition include the enzymes described in Table 1.

TABLE 1

| Condition | Enzyme name | EC number | Principal reaction capable of being catalyzed |
|---|---|---|---|
| (I) | Citrate synthase | 2.3.3.1 | Oxaloacetic acid + acetyl CoA + H₂O ↔ citric acid + |
| | | | CoA |
| (II) | Oxaloacetate decarboxylase | 4.1.1.3 | Oxaloacetic acid ↔ pyruvic acid + CO₂ |
| (III) | Succinate dehydrogenase | 1.3.51 | Quinone + succinic acid ↔ hydroquinone + fumaric |
| | Fumarate reductase | 1.3.5.4 | acid |
| | | 1.3.16 | Same as above (reverse reaction) |
| | | | Succinic acid + NAD ↔ fumaric acid + NADH |
| (IV) | Lactate dehydrogenase | 1.1.5.12 | (R)-Lactic acid + quinone ↔ pyruvic acid + quinol |
| | | 1.1.1.27 | (S)-Lactic acid + NAD⁺ ↔ pyruvic acid + NADH + H⁺ |
| | | 1.1.1.28 | (R)-Lactic acid + NAD⁺ ↔ pyruvic acid + NADH + H⁺ |
| | | 1.1.2.4 | (R)-Lactic acid + 2[Fe(III) cytochrome c] ↔ 2[Fe(II) |
| | | | cytochrome c] + 2H⁺ + pyruvic acid |
| | | 1.1.2.3 | (S)-Lactic acid + 2[Fe(III) cytochrome c] ↔ 2[Fe(II) |
| | | | cytochrome c] + 2H⁺ + pyruvic acid |
| (VI) | Pyruvate:quinone | 1.2.5.1 | Pyruvic acid + ubiquinone + H₂O ↔ acetic acid + CO₂ + |
| | oxidoreductase | | ubiquinol |
| | (Pyruvate dehydrogenase | | |
| | (quinone)) | | |
| (VII) | Pyruvate formate lyase | 2.3.1.54 | Acetyl-CoA + formic acid ↔ CoA + pyruvic acid |
| | (formate acetyltransferase) | | |

The satisfaction of each condition may be realized by using various genetic engineering methods. For example, for the citrate synthase gene (gltA), the oxaloacetate decarboxylase gene (odx), the succinate dehydrogenase gene (sdh), the fumarate reductase gene (frd), the lactate dehydrogenase gene (ldh), the pyruvate: quinone oxidoreductase gene (poxB), or the pyruvate formate lyase gene (formate acetyltransferase gene) (plf), a method for gene disruption or promoter disruption that targets these genes or promoters in the genome, changing to a low-expression promoter, or introducing a mutation, or a method for antisense inhibition (antisense RNA) at the mRNA expression level can be adopted. Alternatively, genetic manipulation may be performed to express a peptide or a protein that inhibits each enzyme activity. Alternatively, in a case where the enzyme protein capable of imparting each enzyme activity requires a process of activation by a predetermined endogenous activator in order to exhibit each enzyme activity in the microorganism, the endogenous activator may be inactivated to suppress the exhibition of each enzyme activity and to realize the satisfaction of each condition.

In terms of being able to realize each of the above conditions relatively easily and more reliably, it is preferable to use a method for gene disruption or introducing mutation. More specifically, it is preferable to adopt any of the following embodiments (1) to (6).
(1) An embodiment in which in the genome (chromosomal DNA) of the genetically modified microorganism, an enzyme gene coding region capable of imparting each enzyme activity is completely or partially disrupted, thereby satisfying the condition (I) and/or the condition (II), and optionally any one or more of the conditions (III), (IV), and (VI).
(2) An embodiment in which in the genome of the genetically modified microorganism, a gene expression regulation region (for example, a promoter region) present upstream of the enzyme gene coding region capable of imparting each enzyme activity is completely or partially disrupted, thereby satisfying the condition (I) and/or the condition (II), and optionally any one or more of the conditions (III), (IV), and (IV).
(3) An embodiment in which in the genome of the genetically modified microorganism, the gene expression regulation region (for example, a promoter region) present upstream of the enzyme gene coding region capable of imparting each enzyme activity is changed to a gene expression regulation region that reduces the expression of the enzyme gene, thereby satisfying the condition (I) and/or the condition (II), and optionally any one or more of the conditions (III), (IV), and (IV).
(4) An embodiment in which in the genome of the genetically modified microorganism, a nucleotide mutation that induces one or more amino acid mutations is introduced into each of the enzyme gene coding regions capable of imparting each enzyme activity, thereby satisfying the condition (I) and/or the condition (II), and optionally any one or more of the conditions (III), (IV), and (IV). Here, the “one or more amino acid mutations” means amino acid mutations that may cause a reduction or inactivation of each enzyme activity.
(5) An embodiment in which the endogenous activator that activates the enzymatic activity of the enzyme protein capable of imparting each enzymatic activity is inactivated or reduced by one or more methods according to the above-described embodiments (1) to (4), thereby satisfying the condition (I) and/or the condition (II), and optionally any one or more of the conditions (III), (IV), and (IV).

The embodiments (1) to (5) may be independently adopted in order to realize the reduction or inactivation of each enzyme activity defined by each condition. In addition, in order to satisfy one condition, two or more of the embodiments (1) to (5) may be adopted. For example, in order to satisfy the condition (I), the embodiments (1) and (2) may be adopted. For example, in order to satisfy the condition (I), both the coding region of the citrate synthase gene and the gene expression regulation region may be disrupted. The embodiment (2) may be adopted in order to satisfy the condition (I), and the embodiment (1) may be adopted in order to satisfy any of the condition (II), the condition (III), the condition (IV), the condition (VI), or the condition (VII).

In a case where the embodiment (1) is adopted, in a case where multiple copies of the enzyme gene coding region are present, all of the multiple enzyme gene coding regions may be disrupted, or only a part thereof may be disrupted. By disrupting only a part of the multiple enzyme gene coding regions, the expression of the enzyme gene can be reduced without completely stopping the expression of the enzyme gene. The expression level of the enzyme gene may be adjusted according to the degree of disruption of the multiple enzyme gene coding regions such that the production efficiency of aspartic acid is improved. By homologous recombination or the like described later, the disruption of the enzyme gene coding region may be realized by partial deletion or may be realized by complete deletion.

In a case where the embodiment (2) is adopted, in a case where there are a plurality of gene expression regulation regions upstream of the enzyme gene coding region, all of the plurality of gene expression regulation regions may be disrupted, or only a part thereof may be disrupted. For example, in a case where there are a plurality of promoter regions controlling the expression of the enzyme gene upstream of the enzyme gene coding region, all of the plurality of promoter regions may be disrupted, or only a part thereof may be disrupted. By disrupting only a part of the promoter region, the expression of the enzyme gene can be reduced without completely stopping the expression of the enzyme gene. The expression level of the enzyme gene may be adjusted according to the degree of disruption of the gene expression regulation region such that the production efficiency of aspartic acid is improved. By homologous recombination or the like described later, the disruption of the gene expression regulation region may be realized by partial deletion or may be realized by complete deletion.

In a case where the embodiment (3) is adopted, in a case where there are a plurality of gene expression regulation regions upstream of the enzyme gene coding region, all of the plurality of gene expression regulation regions may be changed, or only a part thereof may be changed. For example, in a case where there are a plurality of promoter regions controlling the expression of the enzyme gene upstream of the enzyme gene coding region, all of the plurality of promoter regions may be changed, or only a part thereof may be changed. The expression level of the enzyme gene may be adjusted according to the degree of change of the gene expression regulation region such that the production efficiency of aspartic acid is improved. The change of the gene expression regulation region may be realized by homologous recombination or the like described later.

In order to satisfy the condition (I), it is preferable to adopt the embodiment (2). For example, in the bacterium of the genus Corynebacterium, the citrate synthase gene has two promoters (a promoter P1 and a promoter P2). In order to satisfy the condition (I), both the promoter P1 and the promoter P2 may be disrupted, or only any one of the promoter P1 and the promoter P2 may be disrupted. In a preferred embodiment, in order to satisfy the condition (I), only the promoter P1 is disrupted, and the disruption is preferably performed by completely deleting the promoter P1.

The disruption of the gene coding region or the gene expression regulation region (hereinafter, collectively referred to as a “target region”) in the genetically modified microorganism can be performed by a known method. Examples of a method for disrupting the target region include a homologous recombination method, a genome editing technique (CRISPR/CAS system), a transposon method, a mutation introduction method, and the like. From the viewpoint that the disruption of the target region can be achieved relatively inexpensively and efficiently, the homologous recombination method is preferable. Examples of the target region disruption method by homologous recombination will be shown below, but the present invention is not limited thereto.

### (Target Region Disruption Method/Target Region Replacement Method by Homologous Recombination)

(1) Determination of target region and cloning of target region

The entire genome sequences of many bacteria such as the genus Corynebacterium, the genus Escherichia, the genus Bacillus, and the genus Clostridium, and various fungi such as Saccharomyces cerevisiae and Yarrowia lipolytica have been determined, and the nucleotide sequences thereof and the amino acid sequences of proteins encoded by the each gene are also known.

For example, in Corynebacterium glutamicum, the entire genome sequence has been determined in a large number of bacterial strains such as ATCC13032 strain, R strain, ATCC21831 strain, and ATCC14067 strain.

The entire genome sequence has also been determined for bacterial strains of the genus Corynebacterium such as Corynebacterium efficiens YS-314 strain; Corynebacterium callunae DSM20147 strain; Corynebacterium ammoniagenes DSM20306 strain; Corynebacterium marinum DSM44953 strain; Corynebacterium humireducens NBRC106098 strain (DSM45392 strain); Corynebacterium halotolerans YIM70093 strain (DSM44683 strain); Corynebacterium deserti GIMN1.010 strain; Corynebacterium maris DSM45190 strain; and Corynebacterium doosanense CAU212 strain (DSM45436 strain).

Even though the entire genome sequence has not been determined, microorganisms in which the gene sequence of each enzyme having each enzyme activity according to the above-described conditions and the amino acid sequence of the enzyme are known are also present.

These known nucleotide sequences and amino acid sequences can be easily obtained from various databases such as a database (https://www.ncbi.nlm.nih.gov/) made available on the Internet by the National Center for Biotechnology Information Support Center (NCBI) (8600 Bethesda Rockville Pike, Maryland, USA).

Table 2 shows information on genes and the like that can be a target for satisfying the condition (I) and the condition (II) in Corynebacterium glutamicum ATCC13032 strain. Information on genes and the like that can be a target in order to satisfy each condition can be obtained from various databases according to the type of microorganisms.

TABLE 2

| Microorganism | Gene symbol | | |
|---|---|---|---|
| (scientific name) | (homolog gene) | Gene ID | Information of GenBank ID (NCBI) |
| Corynebacterium | gltA | NCgl0795 | Coding region: 877838 . . . 879151 |
| glutamicum | (Cgl0829) | | protein_id: WP_011013914.1 |
| ATCC13032 | | | Promoter P1 (−10) region: |
| (genome sequence | | | 877704 . . . 877709 |
| ID: NC_003450.3) | | | Promoter P2 (−10) region: |
| | | | 877467 . . . 877472 |
| | odx | NCgl1241 | Coding region: 1358259 . . . 1359065 |
| | (Cgl1290) | | protein_id: WP_003861462.1 |
| Escherichia coli | gltA | 945323 | Coding region: complementary strand |
| (genome sequence | | | (753185 . . . 754468) |
| ID: NC_000913.3) | | | protein_id: NP_415248.1 |
| | eda | 946367 | Coding region: complementary strand |
| | | | (1932115 . . . 1932756) |
| | | | protein_id: NP_416364.1 |
| Bacillus subtilis | gltB | locus tag: | Coding region: complementary strand |
| (genome sequence | | KS08_09475 | (1933768 . . . 1938327) |
| ID: NZ_CP009748.1) | | | protein_id: WP_014470320.1 |
| Bacillus subtilis | gltA | locus tag: | Coding region: complementary strand |
| (genome sequence | | BSU18450 | (2010070 . . . 2014632) |
| ID: NC_000964.3) | | | protein_id: NP_389727.2 |

Tables 3 to 11 show information on genes and the like that can be a target for satisfying the conditions (III). (IV), and (VI) in various microorganisms, but the present invention is not limited thereto.

TABLE 3

| Microorganism | Gene symbol | | |
|---|---|---|---|
| (scientific name) | (homolog gene) | Gene ID | Information of GenBank ID (NCBI) |
| Corynebacterium | sdhC | 1021096 | Coding region: 392705 . . . 393478 |
| glutamicum | (Cg10370) | | protein_id: NP_599618.1 |
| ATCC13032 | sdhA | 1021051 | Coding region: 393495 . . . 395516 |
| (genome sequence | (Cg10371) | | protein_id: NP_599619.1 |
| ID: NC_003450.3) | sdhB | 1021416 | Coding region: 395516 . . . 396265 |
| | (Cg10372) | | protein_id: NP_599620.1 |
| | ldh | 1020853 | Coding region: complementary strand |
| | (Cg12911) | | (3112447 . . . 3113391) |
| | | | protein_id: NP_602100.1 |
| | poxB | 1020557 | Coding region: complementary strand |
| | (pqo) | | (2776766 . . . 2778505) |
| | (Cg12610) | | protein_id: NP_601811.1 |
| Escherichia coli | sdhC | 945316 | Coding region: 755177 . . . 755566 |
| str. K-12 | | | protein_id: NP_415249.1 |
| substr. MG 1655 | sdhD | 945322 | Coding region: 755566 . . . 755907 |
| (genome sequence | | | protein_id: NP_415250.1 |
| ID: NC_000913.3) | sdhA | 945402 | Coding region: 755907 . . . 757673 |
| | | | protein_id: NP_415251.1 |
| | sdhB | 945300 | Coding region: 757689 . . . 758405 |
| | | | protein_id: NP_415252.1 |
| | ldhA | 946315 | Coding region: complementary strand |
| | | | (1441854 . . . 1442843) |
| | | | protein_id: NP_415898.1 |
| | | | EC number = 1.1.1.28 |
| | dld | 946653 | Coding region: 2222185 . . . 2223900 |
| | | | protein_id: NP_416637.1 |
| | | | EC number = 1.1.5.12 |
| | 11dD | 948121 | Coding region: 3779827 . . . 3781017 |
| | | | protein_id: NP_418062.1 |
| | poxB | 946132 | Coding region: complementary strand |
| | | | (909331 . . . 911049) |
| | | | protein_id: NP_415392.1 |
| | frdD | 948668 | Coding region: complementary strand |
| | (ECK4147) | | (4379007 . . . 4379366) |
| | | | product: fumarate reductase membrane |
| | | | protein FrdD protein_id: NP_418575.1 |
| | | | EC number = 1.3.5.4 |
| | frdC | 948680 | Coding region: complementary strand |
| | (ECK4148) | | (4379377 . . . 4379772) |
| | | | product: fumarate reductase membrane |
| | | | protein FrdC protein_id: NP_418576.1 |
| | | | EC number = 1.3.5.4 |
| | frdB | 948666 | Coding region: complementary strand |
| | (ECK4149) | | (4379783 . . . 4380517) |
| | | | product: fumarate iron-sulfur protein |
| | | | protein_id: NP_418577.1 |
| | | | EC number = 1.3.5.4 |
| | frdA | 948667 | Coding region: complementary strand |
| | (ECK4150) | | (4380510 . . . 4382318) |
| | | | product: fumarate reductase |

TABLE 4
(Subsequent to Table 3)

| | | | |
|---|---|---|---|
| Escherichia coli str. | | | flavoprotein subunit |
| K-12 substr. MG 1655 | | | protein_id: NP_418578.1 |
| (genome sequence | | | EC number = 1.3.5.4 |
| ID: NC_000913.3) | pflB | 945514 | Coding region: complementary strand |
| | (ECK0894; pfl) | | (951272 . . . 953554) |
| | | | product: pyruvate formate-lyase |
| | | | protein_id: NP_415423.1 |
| | | | EC number = 2.3.1.54 |
| | ybiW | 945444 | Coding region: complementary strand |
| | (ECK0813; pflF) | | (860174 . . . 862606) |
| | | | product: putative pyruvate formate lyase |
| | | | protein_id: NP_415344.1 |
| | pflD | 948454 | Coding region: complementary strand |
| | (ECK3942; yijL) | | (4143995 . . . 4146292) |
| | | | product: putative formate acetyltransferase 2 |
| | | | protein_id: NP_418386.1 |
| | tdcE | 947623 | Coding region: complementary strand |
| | (ECK3103; yhaS) | | (3260124 . . . 3262418) |
| | | | product: 2-ketobutyrate formate-lyase/pyruvate |
| | | | formate-lyase 4 |
| | | | protein_id: YP_026205.1 |
| | pflA | 945517 | Coding region: complementary strand |
| | (act; ECK0893) | | (950340 . . . 951080) |
| | | | product: pyruvate formate-lyase activating enzyme |
| | | | protein_id: NP_415422.1 |
| | | | EC number = 1.97.1.4 |
| | pflC | 948453 | Coding region: complementary strand |
| | (ECK3943; yijM) | | (4146258 . . . 4147136) |
| | | | product: putative pyruvate formate-lyase 2 activating |
| | | | enzyme PflC |
| | | | protein_id: NP_418387.3 |
| Bacillus subtilis | sdhC | | Complementary strand (2665158 . . . 2665766) |
| ATCC13952 | | | protein_id: WP_003152571.1 |
| (genome sequence | sdhA | - | Complementary strand (2663364 . . . 2665124) |
| ID: NZ_CP009748.1) | | | protein_id: WP_013353100.1 |
| | sdhB | - | Complementary strand (2662603 . . . 2663361) |
| | | | protein_id: WP_013353099.1 |
| | KS08_RS01490 | - | 282196 . . . 283149 |
| | | | protein_id: WP_014471413.1 |
| | | | L-lactase dehydrogenase |

TABLE 5

| Microorganism | Gene symbol | | |
|---|---|---|---|
| (scientific name) | (homolog gene) | Gene ID | Information of GenBank ID (NCBI) |
| Saccharomyces | SDH1 | 853709 | Chromosome 11 |
| cerevisiae | | | Genome sequence ID: NC_001136.10 |
| S288C | | | Complementary strand (<169207 . . . >171129) |
| | | | transcript_id: NM_001179714.1 |
| | | | protein_id: NP_012774.1 |
| | | | Flavoprotein subunit |
| | SDH2 | 850685 | Chromosome 12 |
| | | | Genome sequence ID: NC_001144.5 |
| | | | Complementary strand (<53131 . . . >53931) |
| | | | transcript_id: NM_001181861.1 |
| | | | protein_id: NP_013059.1 |
| | | | Iron-sulfur protein subunit |
| | SDH3 | 853716 | Chromosome 11 |
| | | | Genome sequence ID: NC_001143.9 |
| | | | <179667 . . . >180263) |
| | | | transcript_id: NM_001179707.1 |
| | | | protein_id: NP_012781.1 |
| | | | Cytochrome b subunit |
| | SDH4 | 851758 | Chromosome 4 |
| | | | Genome sequence ID: NC_001136.10 |
| | | | <817950 . . . >818495) |
| | | | transcript_id: NM_001180485.1 |
| | | | protein_id: NP_010463.1 |
| | | | Membrane anchor subunit |
| | SDH5 | 854083 | Chromosome 15 |
| | | | Genome sequence ID: NC_001147.6 |
| | | | <196507 . . . >196995) |
| | | | transcript_id: NM_001183326.1 |
| | | | protein_id: NP_014570.1 |
| | | | Protein required for flavination of Sdhlp (promoting |
| | | | FAD coupling factor binding required for assembly and |
| | | | activity exhibition of SDH by binding to Sdhlp) |
| | SDH6 | 851986 | Chromosome 4 |
| | | | Genome sequence ID: NC_001136.10 |
| | | | Complementary strand (1233278 . . . 1233517) |
| | | | transcript_id: NM_001184471.3 |
| | | | protein_id: NP_076888.3 |
| | | | Mitochondrial protein related to assembly of SDH |
| | | | (related to maturation of Sdh2p subunit) |
| | SDH7 | 852123 | Chromosome 4 |
| | | | Genome sequence ID: NC_001136.10 |
| | | | <1470017 . . . >1470418) |
| | | | transcript_id: NM_001180819.3 |
| | | | protein_id: NP_010799.3 |

TABLE 6
(Subsequent to Table 5)

| | | | |
|---|---|---|---|
| Saccharomyces | | | Mitochondrial protein related to assembly of SDH |
| cerevisiae S288C | | | (related to maturation of Sdh2p subunit) |
| | SDH8 | 852572 | Chromosome 2 |
| | | | Genome sequence ID: NC_001134.8 |
| | | | Complementary strand (<742160 . . . >742576) |
| | | | transcript_id: NM_001178617.1 |
| | | | protein_id: NP_009828.2 |
| | | | Protein required for assembly of SDH |
| | DLD1 | 851380 | Chromosome 4 |
| | | | Genome sequence ID: NC_001136.10 |
| | | | Complementary strand (<145826 . . . >147589) |
| | | | transcript_id: NM_001180234.1 |
| | | | protein_id: NP_010107.1 |
| | | | Principal D-lactate dehydrogenase |
| | DLD2 | 851376 | Chromosome 4 |
| | | | Genome sequence ID: NC_001136.10 |
| | | | transcript_id: NM_001180238.1 |
| | | | protein_id: NP_010103.1 |
| | | | Secondary D-lactate dehydrogenase |
| | DLD3 | 856638 | Chromosome 4 |
| | | | Genome sequence ID: NC_001137.3 |
| | | | <16355 . . . >17845 |
| | | | transcript_id: NM_001178886.1 |
| | | | protein_id: NP_010843.1 |
| | | | Secondary D-lactate dehydrogenase |

TABLE 7

| Microorganism | Gene symbol | |
|---|---|---|
| (scientific name) | (homolog gene) | Information of GenBank ID (NCBI) |
| Yarrowia lipolytica | SDH1 | Chromosome 1D |
| CLIB89 (W29) | | Genome sequence ID: CP017556.1 |
| | | Complementary strand (1423938 . . . 1426073) |
| | | protein_id: AOW03921.1 |
| | | Flavoprotein subunit 2 |
| | SDH2 | Chromosome 1D |
| | | Genome sequence ID: CP017556.1 |
| | | 3025537 . . . 3026343 |
| | | protein_id: AOW04521.1 |
| | | Iron-sulfur protein subunit |
| | SDH3 | Chromosome 1E |
| | | Genome sequence ID: CP017557.1 |
| | | 3508208 . . . 3509167 |
| | | mRNA: join (3508208 . . . 3508241, 3508686 . . . 3509167) |
| | | protein_id: AOW06147.1 |
| | SDH4 | Chromosome 1A |
| | | Genome sequence ID: CP017553.1 |
| | | Complementary strand (1471620 . . . 1472117) |
| | | protein_id: AOW00655.1 |
| | | SDH2P membrane anchor subunit |
| | SDH5 | Chromosome 1F |
| | | Genome sequence ID: CP017558.1 |
| | | 1579326 . . . 1579763 |
| | | protein_id: AOW07023.1 |
| | | Flavin factor of SDH |
| | DLD1 | Chromosome 1E |
| | | Genome sequence ID: CP017557.1 |
| | | Complementary strand (385330 . . . 387066) |
| | | protein_id: AOW04885.1 |
| | YALI1_E25400g | Chromosome 1E |
| | | Genome sequence ID: CP017557.1 |
| | | 2540045 . . . 2541166 |
| | | protein_id: AOW05746.1 |
| | | Predictive lactate dehydrogenase cytochrome b |

TABLE 8

| Coryneform | | |
|---|---|---|
| bacterial species | Gene symbol | |
| (scientific name) | (homolog gene) | Information of GenBank ID (NCBI) |
| Corynebacterium | sdhC | Genome sequence ID: NC_004369.1 |
| efficiens | | Coding region: 420459-421232 |
| YS-314 | | protein_id: WP_011074957.1 |
| | sdhA | Genome sequence ID: NC_004369.1 |
| | | Coding region: 421253-423265 |
| | | protein_id: WP_006770371.1 |
| | sdhB | Genome sequence ID: NC_004369.1 |
| | | Coding region: 423265-424014 |
| | | protein_id: WP_006770370.1 |
| | ldh | Genome sequence ID: NC_004369.1 |
| | | Coding region: complementary strand |
| | | (2936722-2937675) |
| | | protein_id: WP_035109376.1 |
| | poxB | Uncertain |
| Corynebacterium | sdhC | Genome sequence ID: CP004354.1 |
| callunae | | Coding region: 374848-375621 |
| DSM20147 | | protein_id: AGG65781.1 |
| | sdhA | Genome sequence ID: CP004354.1 |
| | | Coding region: 375641-377653 |
| | | protein_id: AGG65782.1 |
| | sdhB | Genome sequence ID: CP004354.1 |
| | | Coding region: 377653-378402 |
| | | protein_id: AGG65783.1 |
| | ldh | Genome sequence ID: CP004354.1 |
| | | Coding region: 2672590-2673537 |
| | | protein_id: AGG67879.1 |
| | poxB | Genome sequence ID: CP004354.1 |
| | | Coding region: complementary strand |
| | | (2370666-2372405) |
| | | protein_id: AGG67621.1 |
| Corynebacterium | sdhC | Genome sequence ID: CP009244.1 |
| ammoniagenes | | Coding region: 370172-370927 |
| DSM20306 | | protein_id: APT81721.1 |
| | sdhA | Genome sequence ID: CP009244.1 |
| | | Coding region: 370962-37297 |
| | | protein_id: APT81722.1 |
| | sdhB | Genome sequence ID: CP009244.1 |
| | | Coding region: 372977-373726 |
| | | protein_id: APT81723.1 |
| | ldh | Genome sequence ID: CP009244.1 |
| | | Coding region: complementary strand |
| | | (2614468-2615415) |
| | | protein_id: APT83593.1 |
| | poxB | Genome sequence ID: CP009244.1 |
| | | Coding region: complementary strand |
| | | (2383050-2384801) |
| | | protein_id: APT83404.1 |

TABLE 9

| | Gene | |
|---|---|---|
| Coryneform | symbol | |
| bacterial species | (homolog | |
| (scientific name) | gene) | Information of GenBank ID (NCBI) |
| Corynebacterium | sdhC | Genome sequence ID: NZ_CP007790.1 |
| marinum | | Coding region: 279105-279860 |
| DSM44953 | | protein_id: WP_042620633.1 |
| | sdhA | Genome sequence ID: NZ_CP007790.1 |
| | | Coding region: 279887-281896 |
| | | protein_id: WP_042620634.1 |
| | sdhB | Genome sequence ID: NZ_CP007790.1 |
| | | Coding region: 281896-282645 |
| | | protein_id: WP_042620635.1 |
| | ldh | Genome sequence ID: NZ_CP007790.1 |
| | | Coding region: complementary strand |
| | | (2470158-2471123) |
| | | protein_id: WP_042622762.1 |
| | poxB | Genome sequence ID: NZ_CP007790.1 |
| | | Coding region: complementary strand |
| | | (2227741-2229486) |
| | | protein_id: WP_042622095.1 |
| Corynebacterium | sdhC | Genome sequence ID: CP005286.1 |
| humireducens | | Coding region: 269560-270315 |
| NBRC106098 | | protein_id: AJE32111.1 |
| (DSM45392) | sdhA | Genome sequence ID: CP005286.1 |
| | | Coding region: 270342-272351 |
| | | protein_id: AJE32112.1 |
| | sdhB | Genome sequence ID: CP005286.1 |
| | | Coding region: 272351-273100 |
| | | protein_id: AJE32113.1 |
| | ldh | Genome sequence ID: CP005286.1 |
| | | Coding region: complementary strand |
| | | (2555308-2556261) |
| | | protein_id: AJE34285.1 |
| | poxB | Genome sequence ID: CP005286.1 |
| | | Coding region: complementary strand |
| | | (2270826-2272571) |
| | | protein_id: AJE34046.1 |
| Corynebacterium | sdhC | Genome sequence ID: NC_020302.1 |
| halotolerans | | Coding region: 413293-414048 |
| YIM70093 strain | | protein_id: WP_015399818.1 |
| (DSM44683 strain) | sdhA | Genome sequence ID: NC_020302.1 |
| | | Coding region: 414079-416088 |
| | | protein_id: WP_015399819.1 |
| | sdhB | Genome sequence ID: NC_020302.1 |
| | | Coding region: 416088-416837 |
| | | protein_id: WP_015399820.1 |
| | ldh | Genome sequence ID: NC_020302.1 |
| | | Coding region: complementary strand |
| | | (2953144-2954118) |
| | | protein_id: WP_015402062.1 |
| | poxB | Genome sequence ID: NC_020302.1 |
| | | Coding region: complementary strand |
| | | (2649819-2651555) |
| | | protein_id: WP_015401811.1 |

TABLE 10

| | Gene | |
|---|---|---|
| Coryneform | symbol | |
| bacterial species | (homolog | |
| (scientific name) | gene) | Information of GenBank ID (NCBI) |
| Corynebacterium | sdhC | Genome sequence ID: NZ_CP009220.1 |
| deserti | | Coding region: 422535-423308 |
| GIMN1.010, | | protein_id: WP_053544030.1 |
| | sdhA | Genome sequence ID: NZ_CP009220.1 |
| | | Coding region: 423330-425354 |
| | | protein_id: WP_053544031.1 |
| | sdhB | Genome sequence ID: NZ_CP009220.1 |
| | | Coding region: 425354-426103 |
| | | protein_id: WP_053544032.1 |
| | ldh | Genome sequence ID: NZ_CP009220.1 |
| | | Coding region: complementary strand |
| | | (2773439-2774383) |
| | | protein_id: WP_053545854.1 |
| | poxB | Genome sequence ID: NZ_CP009220.1 |
| | | Coding region: complementary strand |
| | | (2464577-2466316) |
| | | protein_id: WP_053545632.1 |
| Corynebacterium | sdhC | Genome sequence ID: NZ_CP006764.1 |
| doosanense | | Coding region: 406322-407077 |
| CAU212 | | protein_id: WP_026159285.1 |
| (DSM45436) | sdhA | Genome sequence ID: NZ_CP006764.1 |
| | | Coding region: 407089-409191 |
| | | protein_id: WP_018021407.1 |
| | sdhB | Genome sequence ID: NZ_CP006764.1 |
| | | Coding region: 409191-409940 |
| | | protein_id: WP_018021406.1 |
| | ldh | Genome sequence ID: NZ_CP006764.1 |
| | | Coding region: 1783629-1784582 |
| | | protein_id: WP_018020959.1 |
| | poxB | Genome sequence ID: NZ_CP006764.1 |
| | | Coding region: complementary strand |
| | | (2289478-2291241) |
| | | protein_id: WP_018022699.1 |
| Arthrobacter | sdhB | Genome sequence ID: NZ_CP026514.1 |
| sp. PGP41 | (iron-sulfur | Coding region: complementary strand |
| | subunit) | (1210020-1210802) |
| | | protein_id: WP_104997174.1 |
| | sdhA | Genome sequence ID: NZ_CP026514.1 |
| | (flavoprotein | Coding region: complementary strand |
| | subunit) | (1210805-1212604) |
| | | protein_id: WP_104997175.1 |
| | sdhD | Genome sequence ID: NZ_CP026514.1 |
| | (subunit D) | Coding region: complementary strand |
| | | (1212713-1213204) |
| | | protein_id: WP_104997176.1 |
| | sdhC | Genome sequence ID: NZ_CP026514.1 |
| | (cytochrome | Coding region: complementary strand |
| | b556 | (1213208-1213588) |
| | subunit) | protein_id: WP_104997177.1 |
| | ldh | Genome sequence ID: NZ_CP026514.1 |
| | | Coding region: 4105995-4106942 |
| | | protein_id: WP_104999408.1 |
| | poxB | Uncertain |

TABLE 11

| | Gene | |
|---|---|---|
| Coryneform | symbol | |
| bacterial species | (homolog | |
| (scientific name) | gene) | Information of GenBank ID (NCBI) |
| Micrococcus luteus | sdhB | Genome sequence ID: NC_012803.1 |
| NCTC 2665 | (iron-sulfur | Coding region: complementary strand |
| | subunit) | ((528020-528811) |
| | | protein_id: WP_010079347.1 |
| | sdhA | Genome sequence ID: NC_012803.1 |
| | (flavoprotein | Coding region: complementary strand |
| | subunit) | (528811-530598) |
| | | protein_id: WP_010079346.1 |
| | sdhD | Genome sequence ID: NC_012803.1 |
| | (subunit D) | Coding region: complementary strand |
| | | (530676-531155) |
| | | protein_id: WP_010079345.1 |
| | sdhC | Genome sequence ID: NC_012803.1 |
| | (cytochrome | Coding region: complementary strand |
| | b556 | (531159-531563) |
| | subunit) | protein_id: WP_010079344.1 |
| | ldh | Genome sequence ID: NC_012803.1 |
| | | Coding region: 2304603-2305589 |
| | | protein_id: WP_010079965.1 |
| | poxB | Genome sequence ID: NC_012803.1 |
| | | Coding region: complementary strand |
| | | (293132-294847) |
| | | protein_id: WP_010079539.1 |

The citrate synthase activity, the oxaloacetate decarboxylase activity, the succinate dehydrogenase activity, the fumarate reductase activity, the lactate dehydrogenase activity, the pyruvate: quinone oxidoreductase activity, and the pyruvate formate lyase activity are each enzyme activity exhibited by citrate synthase (GltA), oxaloacetate decarboxylase (Odx), succinate dehydrogenase (Sdh), fumarate reductase (Frd), lactate dehydrogenase (Ldh), pyruvate: quinone oxidoreductase (Pox or Pqo), and pyruvate formate lyase (Pfl) found in wild-type microorganisms, respectively. Each of the proteins according to these enzymes can be encoded by genes represented by gltA, odx, sdhCAB (in some strains, sdhCABD), IdhA, dld, lldD, and the like (a gene encoding an enzyme protein exhibiting lactate dehydrogenase activity), poxB (pqo), pflABCD, and the like.

In bacteria, generally, citrate synthase (GltA) forms a homodimer. In the coryneform bacteria, oxaloacetate decarboxylase (Odx) forms a homodimer.

In bacteria, succinate dehydrogenase (Sdh) is a protein composed of three subunits of a transmembrane protein (subunit C) encoded by the sdhC gene, a flavin protein subunit (subunit A) encoded by the sdhA gene, and an Fe—S protein (subunit B) encoded by the sdhB gene, and in some cases, SdhD (subunit D), and each of the genes encoding these subunits constitutes an operon in the bacterial genome in the case of a prokaryote. Fumarate reductase (Frd) is a complex composed of subunits D, C, B, and A in bacteria, for example, such as Escherichia coli, and is encoded by the frdDCBA gene (operon). Pyruvate formate lyase (Pfl) is a complex composed of subunits A, B, C, and D in bacteria, for example, such as Escherichia coli, and is encoded by a pflABCD gene (operon).

It is preferable to use a microorganism of which the nucleotide sequence and the protein sequence of the target region (the coding region of each enzyme protein, the expression regulation region, and the like) relating to the condition to be satisfied among the conditions (I), (II), (III), (IV), (VI), and (VII) and the peripheral region thereof are known. By referring to these known sequences, it is possible to easily specify a genome region to be disrupted. However, in the preparation of the genetically modified microorganism, as the microorganism that can be used as the starting material, a microorganism in which the enzyme protein coding region or the peripheral region thereof is unknown can also be used.

In a case where a microorganism in which each enzyme protein coding region or the peripheral region thereof is unknown is used, for example, the coding region of each enzyme gene is appropriately cloned by various genetic engineering techniques, and the nucleotide sequence is determined as necessary, a region to be disrupted can be specified and cloned. For example, in a case where the alignment analysis is performed on the known amino acid sequences of the homologous enzyme proteins, a plurality of certain amino acid conserved regions are found. A degenerate primer is designed for each of the amino acid conserved regions found on the N-terminal side and the C-terminal side of the enzyme protein, and a degenerate PCR method is performed using the genomic DNA of the microorganism to be cloned as a template. As a result, it is possible to amplify and clone a part of the coding region of the target enzyme gene. Thereafter, the nucleotide sequence of this partial coding region is appropriately determined. Next, the cloned partial coding region is subjected to gene disruption as a target of gene disruption to prepare a genetically modified microorganism that satisfies the desired condition. On the other hand, in a case of desiring the preparation of a genetically modified microorganism disrupted over the entire region of the full-length coding region of the target enzyme gene and/or the gene expression regulation region in the vicinity thereof, primers may be appropriately designed in the opposite directions in the inside of a part of the coding region of the enzyme gene in which the nucleotide sequence is determined as described above, the full-length coding region of the target enzyme gene and/or the peripheral region thereof may be cloned by a method such as an inverse PCR method, and the nucleotide sequence of these regions may be determined. As another method, the enzyme gene to be disrupted and the peripheral region thereof may be cloned by preparing a gene library of a target microorganism, designing an appropriate probe, and performing various hybridization methods, and the nucleotide sequences thereof may be determined. Furthermore, for microorganism species in which the sequence of a homologous gene is not available, the target enzyme gene may be cloned by the various genetic engineering methods described above after the combination of the protein purification technique and each enzyme activity measurement method according to the related art is used to identify the target enzyme and partially determine the peptide sequence.

(2) Preparation of plasmid vector for target region disruption/replacement and target region (such as enzyme gene coding region/expression regulation region) disruption/replacement by homologous recombination

Next, with regard to the disruption or replacement of the target region (the enzyme gene coding region, the expression regulation region, or the like) according to each of the above-described conditions, a method for preparing a target region disruption strain or a target region replacement strain using the homologous recombination method will be described.

First, a plasmid vector for target region disruption/replacement which is capable of causing homologous recombination with a target region in a genome of a microorganism is prepared.

As an example of such a plasmid vector for target region disruption/replacement, a vector using a plasmid vector obtained by cloning a target region from a genome of a microorganism is included. Examples of the plasmid vector for target region disruption/replacement include a plasmid vector for target region disruption, which is obtained by inserting a drug-resistant gene, such as a kanamycin-resistant gene, into the inside of the target region of the plasmid vector, and a plasmid vector for target region replacement, which is obtained by inserting a replacement sequence (a low expression promoter sequence, an enzyme gene code sequence to which a degradation-inducing peptide code sequence is added, or the like) into the inside of the target region of the plasmid vector; and the like. In such a plasmid vector for target region disruption, on both sides of the drug-resistant gene, there are regions homologous to the region to be disrupted in the genome of the microorganism. Therefore, since homologous recombination occurs between the genome of the microorganism and the plasmid for target region disruption in a form in which the drug-resistant gene is inserted into a region to be disrupted in the genome of the microorganism, it is possible to disrupt the target region. In addition, by adding a drug relating to the drug-resistant gene to the culture medium, it is also possible to efficiently select a gene disruption strain. Furthermore, in the plasmid vector for target region replacement as described above, there are regions homologous to the region to be disrupted in the genome of the microorganism on both sides of the replacement sequence. Therefore, since homologous recombination occurs between the genome of the microorganism and the plasmid for target region replacement in a form in which the replacement sequence is inserted into a region to be replaced in the genome of the microorganism, it is possible to replace the target region with the replacement sequence.

As another example of the plasmid vector for target region disruption, it is also possible to use a plasmid vector containing a fragment in which regions located on both sides of a region to be disrupted in the genome of the microorganism (that is, 5′ upstream and 3′ upstream of the region to be removed from the genome) are tandemly linked. Such a plasmid for disruption can be acquired, for example, by amplifying each of a 5′ upstream region and a 3′ downstream region of a target region by a PCR method and inserting the amplified fragments in a form in which the fragments are tandemly linked into a predetermined site such as a multicloning site of a plasmid vector. Alternatively, the entire region from the 5′ upstream region to the 3′ downstream region of the target region may be amplified by a PCR method and cloned using various plasmid vectors, then a primer in the reverse direction may be designed in the inside of the cloned region, and a plasmid vector for target region disruption, in which the deletion mutation of the target region is introduced, may be prepared by an inverse PCR method.

In the plasmid for target region disruption/replacement, the sequence length of a region homologous to the microbial genome sequence to be disrupted or replaced is not limited as long as it can cause homologous recombination, but is generally about 500 bp or more and preferably about 1,000 bp or more. Furthermore, since the construction work of the plasmid for target region disruption/replacement is simplified in a case of being able to construct using E. coli for cloning, it is convenient that the plasmid has a replication origin of E. coli. Furthermore, the plasmid for target region disruption/replacement is preferably a plasmid in which a replication origin capable of autonomously replicating in a microorganism to be targeted for disruption or replacement is not present. In a case where the plasmid for target region disruption/replacement has a replication origin of the microorganism, it is recommended to transfer the plasmid into the microorganism after removing the replication origin by a restriction enzyme treatment or the like. In addition, as the plasmid for target region disruption/replacement, a combination of a drug-resistant gene capable of performing selection by a drug and a lethal gene capable of performing positive selection, such as a SacB gene that can produce a toxin that inhibits the growth of Gram-negative bacteria in the presence of sucrose, may be used. In a case where such a plasmid for target region disruption/replacement is used, it is possible to isolate a bacterial strain in which homologous recombination has occurred by selection with a drug, and then perform selection by culturing in a culture medium containing sucrose. Since the target region disruption strain or the target region replacement strain, in which the vector portion is eliminated by the second homologous recombination, can be isolated by culturing in a sucrose-containing culture medium, it is possible to efficiently acquire a target region disruption strain or a target region replacement strain.

The transfer of the plasmid vector for target region disruption/replacement into the microorganism is not particularly limited, and a transformation method established according to various microorganisms may be used. For example, in the case of coryneform bacteria, it is preferable to use an electric pulse method (for example, the method described in Van der Rest et al. Appl. Microbiol Biotechnol 52, pp. 541-545, 1999). According to the electric pulse method, it is possible to efficiently transfer a nucleic acid into a coryneform bacterium cell.

Confirmation of the disruption of the target region of the genome in the genetically modified microorganism can be performed by a PCR method, a Southern hybridization method, various enzyme activity measurement methods, or the like.

### (Regarding Condition (V))

The genetically modified microorganism according to the present aspect may further satisfy, as the condition (V), “modified phosphoenolpyruvate carboxylase activity resistant to feedback inhibition by aspartic acid in wild-type phosphoenolpyruvate carboxylase activity, or exogenous phosphoenolpyruvate carboxylase activity that exhibits higher resistance to feedback inhibition by aspartic acid compared with the wild-type phosphoenolpyruvate carboxylase activity exhibited by the wild-type microorganism is provided”.

The meaning of “resistance to feedback inhibition by aspartic acid in the wild-type phosphoenolpyruvate carboxylase activity” in the condition (V) is as described below.

Specifically, the “phosphoenolpyruvate carboxylase activity” refers to an enzyme activity that catalyzes a reaction defined in EC4.1.1.31, and is an enzyme activity exhibited by phosphoenolpyruvate carboxylase (PEPC) widely possessed by various plants and microorganisms. A metabolic reaction catalyzed by PEPC is shown below.

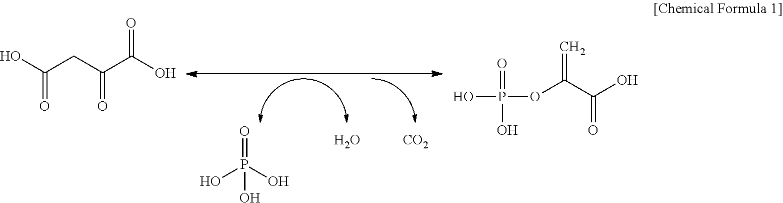

It is known that the enzyme activity of the wild-type phosphoenolpyruvate carboxylase is inhibited with an allosteric effect by metabolites such as aspartic acid, malic acid, and α-ketoglutaric acid (2-oxoglutaric acid), and this inhibition of the enzyme activity is referred to as “feedback inhibition” (Chen Z, et al., Appl Environ Microbiol. 2014 February; 80 (4): 1388-93.; Wada M, et al., J Biosci Bioeng. 2016 February; 121 (2): 172-7.; Yano M, et al., Eur J Biochem. 1997 Jul. 1; 247 (1): 74-81.). That is, the “modified phosphoenolpyruvate carboxylase activity” is defined by an enzyme characteristic in which, in comparison with a corresponding wild-type microorganism or wild-type phosphoenolpyruvate carboxylase possessed by the microorganism, phosphoenolpyruvate carboxylase activity is exhibited and feedback inhibition by aspartic acid is significantly reduced in the enzyme activity.

The meaning of the term “exogenous phosphoenolpyruvate carboxylase activity that exhibits higher resistance to feedback inhibition by aspartic acid compared with the wild-type phosphoenolpyruvate carboxylase activity exhibited by the wild-type microorganism” is as follows.

That is, the above term means the exogenous phosphoenolpyruvate carboxylase activity that exhibits higher resistance to feedback inhibition by aspartic acid compared with “resistance to feedback inhibition by aspartic acid” exhibited by the wild-type phosphoenolpyruvate carboxylase possessed by the wild-type microorganism corresponding to the species to which the genetically modified microorganism belongs, or the wild-type microorganism used as a starting material for preparing the genetically modified microorganism. Such exogenous phosphoenolpyruvate carboxylase activity can be imparted by, specifically, a heterogenous phosphoenolpyruvate carboxylase possessed by a strain line or biological species different from the “corresponding wild-type host microorganism”. The “biological species different from the wild-type host microorganism” includes various biological species such as microorganisms (for example, fungi and prokaryotes such as archaea and bacteria), plants, and animals such as mammals. Furthermore, the imparting of “exogenous phosphoenolpyruvate carboxylase activity” can be realized by, more specifically, transfer of a nucleic acid encoding a PEPC gene isolated from the “strain line or a biological species different from the wild-type host microorganism”.

With regard to the fact that “modified phosphoenolpyruvate carboxylase (activity)” “represents resistance to feedback inhibition by aspartic acid in wild-type phosphoenolpyruvate carboxylase activity” and that “exogenous phosphoenolpyruvate carboxylase (activity)” “represents higher resistance to feedback inhibition by aspartic acid than wild-type phosphoenolpyruvate carboxylase activity exhibited by wild-type microorganisms” can be confirmed by, for example, using a measuring method described in the document (Chen Z, et al., Appl. Environ. Microbiol. 2014 February; 80 (4): 1388-93.; Wada M, et al., J. Biosci. Bioeng. 2016 February; 121 (2): 172-7.; Yano M, et al., Eur. J. Biochem. 1997 Jul. 1; 247 (1): 74-81.), a measuring method described in Yoshinaga T, Izui K, and Katsuki H, J. Biochem., 68, 747-750 (1970), or the like.

In the present specification, “phosphoenolpyruvate carboxylase” may be represented by “PEPC” or “ppc”.

The satisfaction of the condition (V) is not particularly limited, and may be realized in the following aspect. That is, by introducing an amino acid mutation by a genetic engineering method to the protein sequence of the wild-type phosphoenolpyruvate carboxylase retained by each microorganism, a gene encoding a mutant enzyme that has acquired “resistance to feedback inhibition by aspartic acid in wild-type phosphoenolpyruvate carboxylase activity” while maintaining “phosphoenolpyruvate carboxylase activity” may be prepared. For example, a base replacement technique such as a random mutation introduction method by an error-prone PCR or a PCR-based site-specific mutation introduction method using a mutation primer may be used. A more advantageous mutant PEPC may be prepared by applying a molecular evolution method such as DNA shuffling to a plurality kinds of wild-type PEPC coding DNAs.

The nucleic acid encoding the mutant PEPC acquired as described above can be transferred into various microorganisms to prepare a genetically modified microorganism satisfying (V). More specifically, the nucleic acid encoding the mutant PEPC may be transferred into various microorganisms in a form in which the mutant PEPC can be expressed. In the technical field, a gene expression system suitable for each microbial species has already been established in many microbial species including coryneform bacteria. For the microorganism in which the technique according to the known gene expression system can be used, a known technique may be used to introduce the mutant PEPC into the microorganism. Genetic modification techniques or gene expression system techniques may be independently developed and the techniques may be used for introducing the mutant PEPC into a microorganism.

The mutant PEPC that satisfies the condition (V) is not particularly limited, but is preferably a mutant enzyme obtained by introducing a predetermined mutation into the wild-type PEPC derived from bacteria. Such mutant PEPC is a mutant enzyme obtained by introducing a predetermined mutation into a wild-type PEPC preferably derived from coryneform bacteria and more preferably the bacterium of the genus Corynebacterium.

Examples of available bacterial-derived PEPC are shown in Table 12.

TABLE 12

| | | SEQ ID NO: |
|---|---|---|
| Source (bacterium type) | Information of GenBank ID (NCBI) | (protein sequence) |
| Corynebacterium glutamicum | Gene ID: 101955 | 2 |
| ATCC13032 | Genome sequence ID: NC_003450.3 | |
| | Coding region: complementary strand | |
| | (1677384-1680143) | |
| | Gene symbol: Cg11585 | |
| | protein_id: NP_600799.1 | |
| Corynebacterium efficiens | Genome sequence ID: NC_004369.1 | 3 |
| YS-314 | Coding region: complementary strand | |
| | (1792342-1795101) | |
| | locus_tag: CE_RS08485 | |
| | protein_id: WP_006767704.1 | |
| Corynebacterium callunae | Genome sequence ID: CP004354 | 4 |
| DSM20147 | Coding region: complementary strand | |
| | (11594895-1597654) | |
| | protein_id: AGG66941.1 | |
| Corynebacterium ammoniagenes | Genome sequence ID: CP009244.1 | 5 |
| DSM20306 | Coding region: complementary strand | |
| | (486124-488886) | |
| | protein_id: APT81814.1 | |
| Corynebacterium marinum | Genome sequence ID: NZ_CP007790.1 | 6 |
| DSM44953 | Coding region: complementary strand | |
| | (1359285-1362056) | |
| | protein_id: WP_042621481.1 | |
| Corynebacterium humireducens | Genome sequence ID: CP005286.1 | 7 |
| NBRC106098 = DSM45392 | Coding region: complementary strand | |
| | (1428883-1431654) | |
| | protein_id: AJE33253.1 | |
| Corynebacterium halotolerans | Genome sequence ID: NC_020302.1 | 8 |
| YIM70093 = DSM44683 | Coding region: complementary strand | |
| | (1664275-1667040) | |
| | protein_id: WP_015400953.1 | |
| Corynebacterium deserti | Genome sequence ID: NZ_CP009220.1 | 9 |
| GIMN1.010 | Coding region: complementary strand | |
| | (1613811-1616594) | |
| | protein_id: WP_082353424.1 | |
| Corynebacterium doosanense | Genome sequence ID: NZ_CP006764.1 | 10 |
| CAU212 = DSM45436 | Coding region: complementary strand | |
| | (263722-266466) | |
| | protein_id: WP_018021559.1 | |
| Corynebacterium pollutisoli | Genome sequence ID: FXAR01000006.1 | 11 |
| VDS | Coding region: complementary strand | |
| | (149213-151984) | |
| | protein_id: SMG31000.1 | |
| Arthrobacter sp. PGP41 | Genome sequence ID: NZ_CP026514.1 | 12 |
| | Coding region: 621955-624777 | |
| | protein_id: WP_104996751.1 | |
| Escherichia coli str. K-12 | Genome sequence ID: NC_010473.1 | 13 |
| substr. DH10B | Coding region: complementary strand | |
| | (4248167-4250818) | |
| | protein_id: WP_001005586.1 | |

Examples of a specific constitution of the mutant PEPC that satisfies the condition (V) include the following embodiments (i) and (ii).
(i) a mutant PEPC in which one or a plurality of amino acids are deleted, substituted, or added with respect to the amino acid sequence of wild-type PEPC. Here, the range of “one or a plurality” is, for example, 1 to 100, 1 to 50, or 1 to 30, preferably at least 2 or more, 2 to 20, more preferably 2 to 10, still more preferably 2 to 5, and particularly preferably 2 to 4 or 2 to 3, and for example, 2.
(ii) a chimeric PEPC composed of a combination of parts of the amino acid sequences of two or more of the wild-type PEPCs.

In the genetically modified microorganism according to some embodiments, a nucleic acid encoding a mutant phosphoenolpyruvate carboxylase derived from a bacterium is transferred in a form capable of expressing the mutant phosphoenolpyruvate carboxylase. The mutant phosphoenolpyruvate carboxylase has at least one amino acid mutation that satisfies the condition (V) for the genetically modified microorganism. The mutant phosphoenolpyruvate carboxylase is preferably a mutant PEPC derived from coryneform bacteria or a bacterium of the genus Corynebacterium, or a bacterium of the genus Escherichia, more preferably a mutant PEPC derived from a bacterium of the genus Corynebacterium, and particularly preferably a mutant PEPC derived from Corynebacterium glutamicum.

In some embodiments, the at least one amino acid mutation in the mutant phosphoenolpyruvate carboxylase includes at least one amino acid substitution selected from the group consisting of the following (a) to (f) based on the amino acid sequence set forth in SEQ ID NO: 2.
(a) Amino acid substitution of an amino acid corresponding to aspartic acid at position 299 with a predetermined amino acid (here, the amino acid after substitution is not aspartic acid, and an amino acid substitution with alanine, asparagine, glycine, or serine is preferable);
(b) amino acid substitution of an amino acid corresponding to lysine at position 653 with a predetermined amino acid (here, the amino acid after the substitution is not lysine, and an amino acid substitution with alanine, asparagine, or serine is preferable);
(c) amino acid substitution of an amino acid corresponding to lysine at position 813 with a predetermined amino acid (here, the amino acid after the substitution is not lysine, and an amino acid substitution t with alanine, asparagine, glycine, or serine is preferable);
(d) amino acid substitution of an amino acid corresponding to serine at position 869 with a predetermined amino acid (here, the amino acid after the substitution is not serine, and an amino acid substitution with alanine, asparagine, or glycine is preferable);
(e) amino acid substitution of an amino acid corresponding to arginine at a position 873 with a predetermined amino acid (here, the amino acid after the substitution is not arginine, and an amino acid substitution with alanine, phenylalanine, glycine, or serine is preferable); and
(f) Amino acid substitution of an amino acid corresponding to asparagine at a position 917 with a predetermined amino acid (here, the amino acid after substitution is not asparagine, and an amino acid substitution with alanine, phenylalanine, glycine, or serine is preferable).

In the above-described (a) to (f), the amino acid before substitution t and the amino acid after substitution are different from each other.

The amino acids shown in (a) to (f) are intended to specify an amino acid substitution site in the PEPC amino acid sequence to be subjected to mutation introduction based on the amino acids included in the amino acid sequence set forth in SEQ ID NO: 2. That is, more specifically, the “corresponding amino acid” in (a) to (f) refers to an amino acid that is aligned one by one with the amino acid shown in (a) to (f) in a case of where the amino acid sequence set forth in SEQ ID NO: 2 is subjected to one by one alignment (pairwise alignment) based on the identity of the PEPC amino acid sequence to be subjected to mutation introduction by the method of ClustalW or ClustalX (Bioinformatics, Volume 23, Issue 21, November 2008, pp. 2947 to 2948; Bioinformatics, Volume 23, Issue 21, Nov. 1, 2007, pp. 2947 to 2948) or the like.

The “amino acid corresponding to the aspartic acid at position 299” in (a) is aspartic acid (D) in all of 9 wild-type PEPC belonging to the genus Corynebacterium, threonine (T) in the wild-type PEPC of Arthrobacter globiformis NBRC12137 strain, which is one of the coryneform bacteria, and glutamic acid (E) in the wild-type PEPC of Escherichia coli K-12 strain. The “amino acid corresponding to the lysine at position 653” in (b) is arginine (R) in C. ammoniagenes and histidine (H) in C. doosanense for the 9 wild-type PEPC belonging to the genus Corynebacterium, but is the same as lysine (K) in the reference sequence in all of the other bacterial species. The “amino acid corresponding to the lysine at position 813” in (c) is the same as lysine (K) in the reference sequence in all bacterial species. The “amino acid corresponding to the serine at position 869” in (d) is the same as serine(S) in the reference sequence in all bacterial species. The “amino acid corresponding to the arginine at position 873” in (e) is the same as arginine (R) in the reference sequence in all bacterial species. The “amino acid corresponding to the asparagine at position 917” in (f) is threonine (T) in C. ammoniagenes, valine (V) in C. doosanense, and asparagine (N) in the reference sequence in all of the other bacterial species.

For example, the amino acid may be represented such that “aspartic acid at position 299” is represented as “D299” using a one-letter notation of an amino acid and “amino acid substitution of aspartic acid at position 299 with asparagine” is represented as “D299N”. Other amino acids and amino acid substitutions can also be represented in the same manner.

In the preferred embodiments, the at least one amino acid mutation in the mutant phosphoenolpyruvate carboxylase includes at least one amino acid substitution selected from the group consisting of the following (g) to (1) based on the amino acid sequence set forth in SEQ ID NO: 2.
(g) amino acid substitution of an amino acid corresponding to aspartic acid at position 299 with aspartic acid;
(h) amino acid substitution of an amino acid corresponding to lysine at position 653 with serine;
(i) amino acid substitution of an amino acid corresponding to lysine at position 813 with a predetermined amino acid (here, the amino acid after the substitution is not lysine, and an amino acid substitution with glycine or serine is preferable);
(j) amino acid substitution of the amino acid corresponding to serine at position 869 with glycine;
(k) amino acid substitution of an amino acid corresponding to arginine at position 873 with glycine; and
(l) amino acid substitution of an amino acid corresponding to asparagine at position 917 with a predetermined amino acid (here, the amino acid after substitution is not asparagine, and an amino acid substitution with alanine, phenylalanine, glycine, or serine is preferable).

In a more preferred embodiment, the at least one amino acid mutation in the mutant phosphoenolpyruvate carboxylase includes the amino acid substitution shown in (g) and at least one of the amino acid substitution shown in (h) to (1).

In another preferred embodiment, the at least one amino acid mutation in the mutant phosphoenolpyruvate carboxylase includes the amino acid substitution shown in (g) and at least one of the amino acid substitutions shown in (i) to (1).

In a still more preferred embodiment, the at least one amino acid mutation in the mutant phosphoenolpyruvate carboxylase includes the amino acid substitution shown in (g) and the amino acid substitution shown in (i) or (1).

In another embodiment, the mutant phosphoenolpyruvate carboxylase may be a mutant PEPC having an amino acid sequence shown in any one of the following (A) to (C).
(A) an amino acid sequence in which at least one amino acid substitution selected from the group consisting of (a) to (1) is introduced in the amino acid sequence set forth in any one of SEQ ID NO: 2 to 13 (preferably SEQ ID NO: 2 to 12, and more preferably SEQ ID NO: 2 to 11) (here, the amino acid before substitution and the amino acid after substitution are different from each other);
(B) an amino acid sequence in which one or a plurality of amino acids are deleted, substituted, and/or added in the amino acid sequence defined in (A) (provided that the at least one amino acid substitution is maintained); and
(C) an amino acid sequence having at least 60% sequence identity to the amino acid sequence defined in (A) (provided that the at least one amino acid substitution is maintained).

In another embodiment, the mutant phosphoenolpyruvate carboxylase may be a mutant PEPC having an amino acid sequence shown in any one of the following (D) to (F).
(D) an amino acid sequence in which the amino acid substitution shown in (g) and at least one of the amino acid substitution shown in (h) to (1) are introduced in the amino acid sequence set forth in any one of SEQ ID NO: 2 to 13 (preferably SEQ ID NO: 2 to 12, and more preferably SEQ ID NO: 2 to 11) (here, the amino acid before substitution and the amino acid after substitution are different from each other);
(E) an amino acid sequence in which one or a plurality of amino acids are deleted, substituted, and/or added in the amino acid sequence defined in (D) (provided that each of the amino acid substitutions is maintained); and
(F) an amino acid sequence having at least 60% sequence identity to the amino acid sequence defined in (D) (provided that each of the amino acid substitutions is maintained).

In another embodiment, the mutant phosphoenolpyruvate carboxylase may be a mutant PEPC having an amino acid sequence shown in any one of the following (G) to (I).
(G) an amino acid sequence in which the amino acid substitution shown in (g) and at least one of the amino acid substitution shown in (i) to (1) are introduced in the amino acid sequence set forth in any one of SEQ ID NO: 2 to 13 (preferably SEQ ID NO: 2 to 12, and more preferably SEQ ID NO: 2 to 11) (here, the amino acid before substitution and the amino acid after substitution are different from each other);
(H) an amino acid sequence in which one or a plurality of amino acids are deleted, substituted, and/or added in the amino acid sequence defined in (G) (provided that each of the amino acid substitutions is maintained); and
(I) an amino acid sequence having at least 60% sequence identity to the amino acid sequence defined in (G) (provided that each of the amino acid substitutions is maintained).

In another embodiment, the mutant phosphoenolpyruvate carboxylase may be a mutant PEPC having an amino acid sequence shown in any one of the following (J) to (L).
(J) an amino acid sequence in which the amino acid substitution shown in (g) and the amino acid substitution shown in (i) or (1) are introduced in the amino acid sequence set forth in any one of SEQ ID NO: 2 to 13 (preferably SEQ ID NO: 2 to 12, and more preferably SEQ ID NO: 2 to 11) (here, the amino acid before substitution and the amino acid after substitution are different from each other);
(K) an amino acid sequence in which one or a plurality of amino acids are deleted, substituted, and/or added in the amino acid sequence defined in (J) (provided that each of the amino acid substitutions is maintained); and
(L) an amino acid sequence having at least 60% sequence identity to the amino acid sequence defined in (J) (provided that each of the amino acid substitutions is maintained).

In (B), (E), (H), and (K), the range of “one or a plurality” is, for example, 1 to 100, 1 to 50, or 1 to 30, preferably 1 to 20, 1 to 15, or 1 to 10, and more preferably 1 to 9, 1 to 8, 1 to 7, 1 to 6, 1 to 5, 1 to 4, 1 to 3, or 1 or 2.

In (C), (F), (I), and (L), “at least 60%” may be read as preferably at least 70%, more preferably at least 80%, and still more preferably at least 85%, 90%, 91%, 92%, 93%, 94%, 95%, 96%, 97%, 98%, or 99%”.

In (A), (D), (G), and (J), an embodiment in which the “amino acid sequence set forth in any one of SEQ ID NOs: 2 to 13 (preferably SEQ ID NOs: 2 to 12, and more preferably SEQ ID NOs: 2 to 11)” is read as the “amino acid sequence set forth in SEQ ID NO: 2” (that is, the amino acid sequence of the wild-type PEPC of Corynebacterium glutamicum ATCC13032 strain) is particularly preferable.

In each of the above-described embodiments, the mutant PEPC having the amino acid sequence defined in any of (A) to (L) is still intended to retain the phosphoenolpyruvate carboxylase activity and satisfy the condition (V).

In some embodiments, the genetically modified microorganism may be a microorganism in which, for example, aspartate dehydrogenase (AspDH, EC 1.4.1.21), aspartate aminotransferase (AspC, EC 2.6.1.1), and the aspartate ammonia lyase (AspA, EC 4.3.1.1) are enhanced, and for the enhancement of these enzyme activities, a gene encoding these enzymes may be additionally introduced. Examples of the enzyme gene transferred into the coryneform bacteria include the enzyme genes as disclosed in JP2010-183860A, JP2016-516435A, and the like. The contents disclosed in these prior art documents are incorporated herein by reference.

According to the genetically modified microorganism in which each of the above-described embodiments is appropriately adopted, it is possible to use a starting substrate such as sugars for more efficiently producing aspartic acid (particularly L-aspartic acid) or a derivative thereof, and a remarkable improvement in the production efficiency of aspartic acid or a derivative thereof can be expected. Therefore, the genetically modified microorganism of each of the above-described embodiments can be used for producing aspartic acid or a derivative thereof. The “derivative of aspartic acid” refers to a compound generated by the metabolism of aspartic acid in the cell of the genetically modified microorganism. Examples of the derivative of aspartic acid include-alanine. B-Alanine is a compound generated by a decarboxylation reaction of L-aspartic acid. The reaction is catalyzed by aspartate-1-decarboxylase.

Furthermore, the genetically modified microorganism of the present aspect has a feature in that the production amount of by-products is small compared with the corresponding wild-type microorganism or the genetically modified microorganism that does not satisfy the condition (I) and/or the condition (II). Therefore, it is possible to obtain an aspartic acid composition or an aspartic acid derivative composition in which the content of by-products is reduced. Thus, the cost of purifying aspartic acid or a derivative thereof can be reduced, and the risk of adverse effects due to by-products is reduced in a case of being used as an industrial raw material.

Examples of the by-products include organic acids, and amino acids other than aspartic acid or amino acids other than aspartic acid and derivatives thereof. Examples of the organic acid as the by-product include lactic acid, succinic acid, malic acid, citric acid, cis-ascot acid, D-isocitric acid, α-ketoglutaric acid, succinyl CoA, and the like. Examples of the amino acid as the by-product include glutamic acid, alanine, and the like. Particularly, examples of the by-product reduced in the genetically modified microorganism of the present aspect include at least one selected from the group consisting of lactic acid, succinic acid, malic acid, glutamic acid, and alanine. In the genetically modified microorganism of the present aspect, the production amounts of lactic acid and alanine are preferably reduced and the production amounts of all of lactic acid, succinic acid, malic acid, glutamic acid, and alanine are more preferably reduced compared with the corresponding wild-type microorganism or a genetically modified microorganism that does not satisfy the condition (I) and/or the condition (II).

### <Method of Producing Aspartic Acid or Derivative Thereof>

A second aspect of the present invention is a method for producing aspartic acid or a derivative thereof, including the following (p) and (q).
(p) Producing aspartic acid or a derivative thereof using the cell of the genetically modified microorganism according to the first aspect or a treated product of the cell; and
(q) recovering the aspartic acid or a derivative thereof.

In step (p), the genetically modified microorganism according to the first aspect may be cultured under an aerobic condition in which the genetically modified microorganism can substantially proliferate, thereby producing aspartic acid or a derivative thereof. Under the aerobic condition, in the coryneform bacteria, the metabolism proceeds clockwise in the TCA cycle shown in FIG. 1. Therefore, in the genetically modified microorganism according to the first aspect, the amount of oxaloacetic acid accumulated is increased, and aspartic acid or a derivative thereof can be efficiently produced.

On the other hand, for example, in a microorganism such as the bacterium of the genus Escherichia such as E. coli, or the coryneform bacterium, in a culture medium or a reaction liquid under a reducing condition, a specific metabolic system under the reducing condition functions without substantial proliferation. Therefore, in a case where the coryneform bacteria or the treated product of the cell is reacted in the culture medium or the reaction liquid under the reducing condition in this way, it is possible to remove the waste of the nutrient source due to the proliferation and division of the cells of the microorganism, thereby the conversion efficiency of the nutrient source into aspartic acid can be improved. In addition, since the genetically modified microorganism according to the first aspect satisfies the condition (I) and/or the condition (II) and optionally satisfies any or all of the conditions (III) to (VII), it is expected that the conversion efficiency of the nutrient source into aspartic acid is remarkably improved. In a case where the reaction proceeds under the reducing condition in which the microorganisms do not substantially proliferate, it is possible to prevent the generation of fermentation heat compared with the bioprocess under the aerobic condition accompanied by the division/proliferation of cells, and it is not necessary to secure sufficient aeration during the culture. Therefore, it is possible to simplify the equipment and reduce the energy required for the bioprocess, which leads to environmental and cost advantages.

Therefore, in step (p), aspartic acid or a derivative thereof may be produced by reacting the cell of the genetically modified microorganism or a treated product of the cell in reaction medium (X) under a reducing condition in which the genetically modified microorganism does not substantially proliferate.

With regard to step (p′):
The method according to the present aspect further may include, before step (p),
(p′) culturing and proliferating the genetically modified microorganism in predetermined culture medium (Y) under an aerobic condition.

The cell of the genetically modified microorganism which has been proliferated in step (p′) or a treated product of the cell may be provided to step (p).

The embodiment including step (p′) is an embodiment assumed in the embodiment relating to the substance production under aerobic conditions, but it is particularly preferably applied in a case where the substance production is carried out in reaction medium (X) under reducing conditions in which the genetically modified microorganism does not substantially proliferate. In a case where, as step (p′), the genetically modified microorganism is proliferated under an aerobic condition to a certain extent in advance, and then, in step (p), a sufficient amount of genetically modified microorganism proliferated is used to advance the substance production in reaction medium (X) in which the genetically modified microorganism does not substantially proliferate, the genetically modified microorganism can be used like a chemical catalyst to efficiently produce a substance. In addition, the genetically modified microorganism can be recovered from reaction medium (X) after the substance production in reaction medium (X) and reused for the reaction of step (p) of the second cycle and subsequent cycles, as necessary.

Specific configurations and elements that can be adopted in these steps will be described below in order of step (p′), step (p), and step (q).

### (Basic Culture Medium Constituting Culture Medium (Y))

Culture medium (Y) is not particularly limited, and may be appropriately selected and used depending on the type of genetically modified microorganism used in the method. Specifically, as culture medium (Y), a natural culture medium or a synthetic culture medium containing a carbon source, a nitrogen source, inorganic salts, other nutrient substances, and the like can be used. The components contained in the culture medium are, for example, as follows.

Examples of the carbon source include carbon-containing substances such as carbohydrates, more specifically, sugars including polysaccharides and monosaccharides, various materials containing these, and the like, and examples thereof include the following components.

Monosaccharides such as glucose, fructose, mannose, xylose, arabinose, and galactose; disaccharides such as sucrose, maltose, lactose, cellobiose, xylobiose, and trehalose; polysaccharides such as cellulose, starch, glycogen, agarose, pectin, and alginic acid; syrup (molasses) and the like; inedible agricultural production waste and inedible biomass (resources obtained from inedible herbaceous plants or woody plants as a raw material) such as rice straw, forest residues, bagasse, and corn stover; a saccharification solution containing a plurality of sugars such as glucose and xylose obtained by saccharifying energy crops such as switchgrass, napiergrass, and miscanthus with a saccharifying enzyme or the like; sugar alcohols such as mannitol, sorbitol, xylitol, and glycerin; organic acids such as acetic acid, citric acid, lactic acid, fumaric acid, maleic acid, and gluconic acid; alcohols such as ethanol, propanol, and butanol; and hydrocarbons such as normal paraffin.

As the carbon source, one may be used alone or two or more may be used in a mixture.

As the nitrogen source, inorganic or organic ammonium compounds such as ammonium carbonate ((NH₄)₂CO₃), ammonium chloride, ammonium sulfate, ammonium nitrate, and ammonium acetate; urea; aqueous ammonia; and inorganic or organic nitrate compounds such as sodium nitrate and potassium nitrate; and the like can be used. In addition, a nitrogen-containing organic compound such as a corn steep liquor, meat extract, a protein hydrolysate (casamino acid, tryptone, peptone, NZ-amine, and the like), and an amino acid can also be used.

As the nitrogen source, one may be used alone or two or more may be used in combination. The concentration of the nitrogen source in the culture medium may be appropriately adjusted according to the conditions such as the type of genetically modified microorganism to be adopted and properties thereof, and the type of nitrogen compound, and is not particularly limited, but may be, for example, about 0.1% to 10% (w/v).

Examples of the inorganic salts include monopotassium phosphate, dipotassium phosphate, magnesium sulfate (hydrate), sodium chloride, iron (II) sulfate heptahydrate, iron (II) nitrate, manganese sulfate, zinc sulfate, cobalt sulfate, calcium carbonate, and the like.

As the inorganic salt, one may be used alone, or two or more may be used in a mixture. The concentration of the inorganic salts in the culture medium may be appropriately adjusted according to the conditions such as the type of genetically modified microorganism to be adopted and properties thereof, and the type of inorganic salts, and is not particularly limited, but may be, for example, about 0.01% to 1% (w/v).

Examples of the other nutrient substances include meat extract, peptone, polypeptone, yeast extract, dried yeast, corn steep liquor, defatted milk powder, a defatted soybean hydrochloric acid hydrolysate, extracts of animal, plant, or microorganism cells, decomposition products thereof, and the like. The concentration of the other nutrient substances in the culture medium may be appropriately adjusted according to the conditions such as the type of genetically modified microorganism and properties thereof, and the type of nutrient substances, and is not particularly limited, but may be, for example, about 0.1% to 10% (w/v).

Vitamins can also be added to culture medium (Y) as necessary. Examples of the vitamins include biotin, thiamine (vitamin B1), pyridoxine (vitamin B6), pantothenic acid, inositol, and the like.

As necessary, an antifoaming agent such as a silicone-based antifoaming agent or a polyether-based antifoaming agent may be added, and various antifoaming agents for a bacterial culture medium are commercially available, and thus these antifoaming agents may be used.

The pH of culture medium (Y) is not particularly limited as long as the genetically modified microorganism to be adopted can grow, but is preferably about 6 to 8.

In a case where the genetically modified microorganism is a coryneform bacteria, as culture medium (Y), an A culture medium (Inui, M. et al., J. Mol. Microbiol. Biotechnol. 7:182-196 (2004)), a BT culture medium (Omumasaba, C.A. et al., J. Mol. Microbiol. Biotechnol. 8:91-103 (2004)), an NA culture medium, or the like can be preferably used.

In this way, the cell or the treated product of the cell is acquired by culturing and proliferating the genetically modified microorganism according to the first aspect in the above-mentioned culture medium (Y), and may be used in step (p).

The culture conditions for the genetically modified microorganism may be appropriately set such that the genetically modified microorganism sufficiently proliferates and a sufficient amount of cells or a treated product of the cells is obtained. For example, the culture can be performed under aerobic conditions at a culture temperature of about 25° C. to 38° C. and a culture time of about 12 hours to 48 hours. The cell stock obtained by freeze-drying or frozen storage is once seeded on a solid culture medium, and a colony or the like confirmed to grow on the solid culture medium is further inoculated on the above-mentioned culture medium (Y), thereby the genetically modified microorganism to be assayed in step (p) can be prepared.

With regard to step (p): In step (p), aspartic acid or a derivative thereof is produced using a cell of the genetically modified microorganism or a treated product of the cell.

The specific form of the “cell or a treated product of the cell” is not particularly limited as long as it is in a state of being able to produce aspartic acid or a derivative thereof.

In some embodiments, in step (p′), after culturing and proliferating the genetically modified microorganism in culture medium (Y), culture medium (Y) containing the genetically modified microorganism may be directly used in step (p) without recovering or separating the genetically modified microorganism from culture medium (Y). Prior to step (p), a carbon source (sugars), a nitrogen source, inorganic salts, vitamins, a reducing agent, or the like may be added to culture medium (Y) containing the genetically modified microorganism acquired in step (p′) as necessary and culture medium (Y) may be used in step (p).

In another embodiment, the genetically modified microorganism cultured and proliferated in culture medium (Y) in step (p′) may be separated and recovered from culture medium (Y), and the obtained cells themselves may be provided to step (p).

Alternatively, a treated product of the cell obtained by subjecting the cell to a predetermined physical or chemical treatment may be provided to step (p). Examples of the method of separating and recovering the genetically modified microorganism from culture medium (Y) include centrifugation, separation using various filters, decantation, and the like. The “treated product of the cell” is not particularly limited as long as it can realize the production reaction of aspartic acid or a derivative thereof in step (p). Examples thereof include a treated product obtained by subjecting the recovered cells to various drug treatments; a treated product obtained by immobilizing the recovered cells on a carrier such as acrylamide, carrageenan, or other appropriate polymers; and the like.

### (Reaction Under Reducing Condition)

### «Composition of Reaction Medium (X)»

In a case where step (p) is performed under the reducing condition in which the genetically modified microorganism does not substantially proliferate, reaction medium (X) may be used. The composition of reaction medium (X) is not particularly limited as long as it realizes reaction medium (X) under the reducing condition, in which the genetically modified microorganism is substantially not allowed to proliferate and the production reaction of aspartic acid by the genetically modified microorganism is allowed to proceed. Reaction medium (X) contains, for example, a carbon source, a nitrogen source, inorganic salts, and the like, and may be a natural medium derived from a living body or the like, or may be an artificially synthesized medium. The components contained in reaction medium (X) are, for example, as follows.

Examples of the carbon source include carbohydrates, more specifically, sugars including polysaccharides and monosaccharides, various materials containing these, and examples thereof include the following components.

Monosaccharides such as glucose, fructose, mannose, xylose, arabinose, and galactose; disaccharides such as sucrose, maltose, lactose, cellobiose, xylobiose, and trehalose; polysaccharides such as cellulose, starch, glycogen, agarose, pectin, and alginic acid; syrup (molasses) and the like; inedible agricultural production waste and inedible biomass (resources obtained from inedible herbaceous plants or woody plants as a raw material) such as rice straw, forest residues, bagasse, and corn stover; a saccharification solution containing a plurality of sugars such as glucose and xylose obtained by saccharifying energy crops such as switchgrass, napiergrass, and miscanthus with a saccharifying enzyme or the like; sugar alcohols such as mannitol, sorbitol, xylitol, and glycerin; organic acids such as acetic acid, citric acid, lactic acid, fumaric acid, maleic acid, and gluconic acid; alcohols such as ethanol, propanol, and butanol; and hydrocarbons such as normal paraffin.

Among these, monosaccharides are preferable, and glucose is more preferable. Sugars (disaccharides, oligosaccharides, and polysaccharides) containing glucose are also preferable. As the carbon source, one may be used alone, or two or more may be used in combination. The concentration of the carbon source in reaction medium (X) is preferably about 1% to 20% (w/v), more preferably about 2% to 10% (w/v), and still more preferably about 2% to 5% (w/v). The concentration of the sugars in reaction medium (X) is, for example, about 1% to 20% (w/v), and more preferably about 2% to 10% (w/v) and still more preferably about 2% to 5% (w/v).

As the nitrogen source, inorganic or organic ammonium compounds such as ammonium carbonate ((NH₄)₂CO₃), ammonium chloride, ammonium sulfate, ammonium nitrate, and ammonium acetate; urea; aqueous ammonia; and inorganic or organic nitrate compounds such as sodium nitrate and potassium nitrate can be used. A nitrogen-containing organic compound such as a corn steep liquor, meat extract, peptone, NZ-amine, a protein hydrolysate, or an amino acid, or the like can also be used.

As the nitrogen source, one may be used alone, or two or more may be used in combination. The concentration of the nitrogen source in the reaction liquid may be appropriately adjusted according to the conditions such as the type of genetically modified microorganism to be used, reaction conditions, and the type of nitrogen compound, and is not particularly limited, but may be adjusted to, for example, about 0.1% to 10% (w/v).

Examples of the inorganic salts include monopotassium phosphate, dipotassium phosphate, magnesium sulfate (hydrate), sodium chloride, iron (II) sulfate heptahydrate, iron (II) nitrate, manganese sulfate, zinc sulfate, cobalt sulfate, calcium carbonate, and the like.

As the inorganic salt, one may be used alone, or two or more may be used in combination. The concentration of the inorganic salts in the reaction liquid may be appropriately adjusted according to the conditions such as the type of genetically modified microorganism to be used, reaction conditions, and the type of inorganic salts, and is not particularly limited, but may be, for example, about 0.01% to 1% (w/v).

Vitamins can also be added to reaction medium (X) as necessary. Examples of the vitamins include biotin, thiamine (vitamin B1), pyridoxine (vitamin B6), pantothenic acid, inositol, and the like.

The pH of reaction medium (X) is not particularly limited as long as it is in a range in which the reaction for producing aspartic acid proceeds, but is generally preferably about 6.0 to 8.0, more preferably 6.5 to 8.0, and for example, about 7.5.

Specific examples of the basic composition of reaction medium (X) include the above-described BT culture medium and the like, and reaction medium (X) can be prepared by appropriately adjusting the concentration of the carbon source (sugars), the concentration of the nitrogen source, the concentration of the inorganic salts, the concentration of the vitamins, and the like as described above, based on the compositions of the culture media.

«Reducing condition»

The reducing condition under which the genetically modified microorganism does not substantially proliferate means that the reaction medium is in a reduced state to the extent that the genetically modified microorganism does not substantially proliferate, as interpreted literally, but more specifically, the reducing condition may be defined by the oxidation-reduction potential of the reaction medium. The oxidation-reduction potential of reaction medium (X) is preferably in a range of about −200 mV to −500 mV, more preferably in a range of about −250 mV to −500 mV, and still more preferably in a range of −300 to 400 mmV.

The oxidation-reduction potential of reaction medium (X) can be measured using an oxidation-reduction potentiometer. Since there is also a commercially available product of the oxidation-reduction potentiometer, these commercially available products may be used for measuring the oxidation-reduction potential of the reaction medium (X).

The reduced state of reaction medium (X) can be estimated by a simple method using a resazurin indicator (in a case of being in a reduced state, the color changes from blue to colorless), but in a case of more accurate control, the reducing state may be measured using an oxidation-reduction potentiometer (for example, ORP Electrodes, manufactured by Broadley-James Ltd.).

The method for preparing reaction medium (X) under the reducing condition can be performed using various methods without particular limitation, and for example, a known method for preparing the following aqueous solution for a reaction solution can be used.

That is, as the solvent of the reaction medium (X), an aqueous solution for a reaction liquid may be used instead of distilled water or the like. As references of the method of preparing the aqueous solution for a reaction liquid, there are, for example, a method of preparing a culture solution for an obligate anaerobic microorganism such as a sulfate reduction microorganism (Pfennig, N. et al., (1981): The dissimilatory sulfate-reducing bacteria, in the Prokaryotes, A Handbook on Habitats Isolation and Identification of Bacteria, Ed. by Starr, M. P. et al., pp. 926-940, Berlin, Springer Verlag.), “Agricultural Chemistry Experiment Book, Vol. 3, Department of Agricultural Chemistry, Faculty of Agriculture, Kyoto University, 26th edition, 1990, published by Sangyo Tosho Co., Ltd.”, and the like, and a desired aqueous solution under a reducing condition can be obtained.

Specifically, an aqueous solution for a reaction liquid under a reducing condition can be obtained by subjecting distilled water or the like to a heating treatment or a reduced pressure treatment to remove a dissolved gas. In this case, by treating distilled water or the like under a reduced pressure of about 10 mmHg or less, preferably about 5 mmHg or less, and more preferably about 3 mmHg or less for about 1 to 60 minutes, preferably about 5 to 40 minutes, dissolved gas, in particular dissolved oxygen, can be removed to create an aqueous solution for a reaction liquid under a reducing condition (anaerobic state).

An aqueous solution for a reaction liquid under a reducing condition can also be prepared by adding an appropriate reducing agent (for example, thioglycolic acid, ascorbic acid, cysteine hydrochloride, mercaptoacetic acid, thiolacetic acid, glutathione, sodium sulfide, or the like).

A method of appropriately combining these methods is also a method of preparing an aqueous solution of a reaction liquid under an effective reducing condition.

It is preferable to maintain the reduced state of reaction medium (X) even during the reaction in step (p). In order to continuously maintain the reduced state of reaction medium (X) during the reaction, it is desirable to prevent mixing of oxygen from the outside of the reaction system as much as possible, and specific examples thereof include a method of sealing the reaction system with an inert gas such as nitrogen gas, carbon dioxide gas, or the like. As a method for more effectively preventing oxygen mixing, in order to efficiently function the metabolic function in the cells of the aerobic bacteria in the middle of the reaction, it may be necessary to appropriately add a solution for maintaining and adjusting pH of the reaction system or various nutrient-dissolved solutions, but in such a case, it is effective to remove oxygen in advance from the solution to be added.

In a case where the method according to the embodiment of the present invention includes step (p′), culture medium (Y) adjusted by subjecting culture medium (Y) in which the genetically modified microorganism has been proliferated by step (p′) to the predetermined operation and/or adding a reducing agent thereto, such that a reducing condition under which the genetically modified microorganism does not substantially proliferate is satisfied, may be used as reaction medium (X) in step (p).

«Reaction conditions»

The reaction temperature in step (p) may be in a range in which aspartic acid or a derivative thereof is produced, and may be appropriately set according to the properties of the genetically modified microorganism to be adopted and the like, and is not particularly limited. Typically, the temperature is about 20° C. to 50° C., preferably about 25° C. to 47° C., and more preferably about 27° C. to 37° C. As long as the temperature is in such a temperature range, aspartic acid or a derivative thereof can be efficiently produced.

The reaction time may be appropriately adjusted such that aspartic acid or a derivative thereof is obtained, and is not particularly limited. However, for example, the reaction time is in a range of about 1 hour to about 7 days, and from the viewpoint of more efficient production of aspartic acid or a derivative thereof, preferably in a range of about 1 hour to about 3 days, and can be set to, for example, about 1 hour to 48 hours.

The reaction may be performed by any of a batch type, a flow-addition type, or a continuous type. Among these, a batch type is preferable.

### (Reaction Under Low Dissolved Oxygen Concentration Condition)

«Composition of reaction medium (X′)»

Step (p) may be performed under a low dissolved oxygen concentration condition. Examples of the low dissolved oxygen concentration condition include a dissolved oxygen concentration of 0.5 mg/L or less. Examples of the low dissolved oxygen concentration condition include a dissolved oxygen concentration of 0.4 mg/mL or less, 0.35 mg/mL or less, 0.3 mg/mL or less, and 0.25 mg/mL or less. Examples of the range of the dissolved oxygen concentration in the low dissolved oxygen concentration condition include 0.001 to 0.5 mg/L, 0.01 to 0.4 mg/L, 0.05 to 0.3 mg/L, 0.05 to 0.25 mg/L, 0.05 to 0.2 mg/L, and 0.1 to 0.2 mg/L. The low dissolved oxygen concentration condition may be defined by a relative value with respect to the saturated dissolved oxygen concentration. For example, in the low dissolved oxygen concentration condition, the dissolved oxygen concentration may be 10 or less, 9 or less, 8 or less, 7 or less, 6 or less, or 5 or less as a relative value in a case where the saturated dissolved oxygen concentration is set to 100. The dissolved oxygen concentration under the low dissolved oxygen concentration condition may be in a range of 0.1 to 10, 0.5 to 9, 0.5 to 8, 0.5 to 7, 0.5 to 6, 0.5 to 5, 0.5 to 4, or 1 to 3 as a relative value in a case where the saturated dissolved oxygen concentration is set to 100. The saturated dissolved oxygen concentration means a saturated concentration of oxygen dissolved in a reaction medium at 1 atm and a reaction temperature. Aeration is performed in the reaction medium at 1 atm and the reaction temperature, the dissolved oxygen concentration is measured with a dissolved oxygen sensor (DO sensor), and a value at a time when the dissolved oxygen concentration is stable can be adopted as the saturated dissolved oxygen concentration.

In a case where step (p) is performed under a low dissolved oxygen condition, aspartic acid may be produced by reacting the genetically modified microorganism or the treated product of the cell in reaction medium (X′) having a low dissolved oxygen concentration. The oxidation-reduction potential of reaction medium (X′) having a low dissolved oxygen concentration may be about −200 mV to −500 mV. Therefore, the reaction under the low dissolved oxygen condition can also be said to be a reaction under the reducing condition. Reaction medium (X′) used under the low dissolved oxygen condition may be the same as reaction medium (X) used under the reducing condition described above. The oxidation-reduction potential of reaction medium (X′) is preferably in a range of about −250 mV to −500 mV and more preferably in a range of −300 to 400 mmV.

The composition of reaction medium (X′) is not particularly limited as long as it advances the production reaction of aspartic acid or a derivative thereof by the genetically modified microorganism. Reaction medium (X′) contains, for example, a carbon source, a nitrogen source, inorganic salts, and the like, and may be a natural medium derived from a living body or the like, or may be an artificially synthesized medium. Examples of the carbon source, the nitrogen source, and the inorganic salts include the same ones as those for reaction medium (X). Reaction medium (X′) may be obtained by removing the reducing agent from reaction medium (X). Reaction medium (X′) may contain antibiotics that inhibit the growth of the genetically modified microorganism as long as the antibiotics do not inhibit the production reaction of aspartic acid by the genetically modified microorganism. Examples of the antibiotics include chloramphenicol.

The pH of reaction medium (X) is not particularly limited as long as it is in a range in which the reaction for producing aspartic acid or a derivative thereof proceeds, but is generally preferably about 6.0 to 8.5, more preferably 6.5 to 8.5, and for example, about 8.

Specific examples of the basic composition of reaction medium (X′) include those containing a carbon source, a nitrogen source, and antibiotics, and more specifically, include those containing glucose, ammonium sulfate, and chloramphenicol.

### «Reaction Conditions>

The reaction temperature is not particularly limited, but can be set to, for example, 15° C. to 50° C., preferably 20° C. to 47° C., more preferably 20° C. to 37° C., and still more preferably 20° C. to 30° C.

The reaction time is not particularly limited, but can be set to, for example, about 1 hour to about 7 days, preferably about 1 hour to about 3 days, and more preferably about 1 hour to 48 hours.

The reaction may be performed by any of a batch type, a flow-addition type, or a continuous type. Among these, the flow-addition type is preferable.

In the reaction under the low dissolved oxygen condition, stirring and/or aeration may be performed in order to consume NADH accumulated in the cell or the treated product of the cell during the reaction. In a case of performing stirring, the stirring speed is not particularly limited, but can be set to, for example, 100 to 800 rpm, preferably 200 to 600 rpm, and more preferably 300 to 500 rpm. In a case of performing aeration, the aeration speed is not particularly limited, but can be set to, for example, 1 to 20 mL/minute, preferably 2 to 15 mL/minute, more preferably 3 to 10 mL/minute, and still more preferably 3 to 8 mL/minute. In a case of performing stirring and/or aeration, the dissolved oxygen concentration of reaction medium (X′) is preferably adjusted to 0.5 mg/L or less. The dissolved oxygen concentration in reaction medium (X′) can be measured using a dissolved oxygen meter. The oxidation-reduction potential of reaction medium (X′) is preferably adjusted to about −250 mV to −500 mV. The oxidation-reduction potential of reaction medium (X′) can be measured using an oxidation-reduction potentiometer.

### (High-Density Condition)

Step (p) may be performed under a high-density condition of the genetically modified microorganism. Step (p) can be performed under a high-density condition in any of the reducing condition and the low dissolved oxygen condition, which are described above. “Under a high-density condition of the genetically modified microorganism” means a state in which the genetically modified microorganism is present at a high density in reaction medium (X) or reaction medium (X′). In the reaction under the high-density condition, for example, the cell turbidity in reaction medium (X) or reaction medium (X′) at OD₆₁₀ can be adjusted to about 150 to 300, preferably about 200 to 250. “OD₆₁₀” means an optical density of a microorganism culture measured at a wavelength of 610 nm.

After the reaction in step (p) is completed, the genetically modified microorganism or the treated product of the cell may be recovered from reaction medium (X) or reaction medium (X′) by an appropriate operation such as centrifugation, and the recovered genetically modified microorganism or the recovered treated product of the cell may be reused to repeat step (p) a plurality of times. In this way, repeating step (p) a plurality of times by reusing the genetically modified microorganism or the treated product of the cell leads to a reduction in production cost and makes it possible to realize efficient production of aspartic acid.

### With Regard to Step (q):

After aspartic acid or a derivative thereof is generated in step (p), as step (q), aspartic acid or a derivative thereof is recovered. The term “recovering aspartic acid or a derivative thereof” is a concept that encompasses recovering aspartic acid or a derivative thereof by collecting a genetically modified microorganism containing aspartic acid or a derivative thereof and/or a culture or reaction medium thereof.

In step (q), aspartic acid or a derivative thereof may be recovered by collecting the genetically modified microorganism containing aspartic acid or a derivative thereof and/or the culture or reaction medium thereof. Furthermore, aspartic acid or a derivative thereof may be recovered by separating and/or purifying aspartic acid or a derivative thereof from a culture or reaction medium (reaction medium (X), reaction medium (X′), or the like), or genetically modified microorganism cells or treated product of the cell, which contains aspartic acid or a derivative thereof.

In the embodiment in which the separation and/or purification process of aspartic acid or a derivative thereof is adopted, in the separation and/or purification process of aspartic acid or a derivative thereof, an appropriate separation/purification technique may be adopted according to the required purity and the like in consideration of the use of aspartic acid or a derivative thereof. Without particular limitation, aspartic acid or a derivative thereof can be recovered by appropriately combining, for example, various crystallization methods; various filtration techniques such as an ultrafiltration method; various chromatography techniques such as ion exchange chromatography, affinity chromatography, hydrophobic chromatography, and reversed-phase chromatography; a concentration method; dialysis; an activated carbon adsorption method; and the like. Since various separation and purification techniques for these substances are known, they may be appropriately used.

Furthermore, the method according to the present aspect may further include a step of optionally washing, drying, pulverizing, powdering or granulating, and/or packaging aspartic acid or a derivative thereof.

In the method according to the present aspect, aspartic acid or a derivative thereof is produced using the genetically modified microorganism according to the first aspect. Therefore, aspartic acid or a derivative thereof can be efficiently produced. Furthermore, the genetically modified microorganism according to the first aspect can reduce the production amount of by-products (amino acids other than aspartic acid, organic acids, and the like) generated during the production of aspartic acid. More specifically, the production amount of at least one selected from the group consisting of lactic acid, succinic acid, malic acid, glutamic acid, and alanine can be reduced. The production amount of lactic acid and alanine can be preferably reduced. More preferably, the production amounts of all of lactic acid, succinic acid, malic acid, glutamic acid, and alanine can be reduced. In the present specification, the term “alanine” refers to “α-alanine” unless otherwise specified.

For example, in the method of the present aspect, in a case where the genetically modified microorganism satisfying condition (I) is used, the relative production amount of lactic acid can be reduced to 0.9 or less, 0.8 or less, 0.7 or less, 0.6 or less, 0.5 or less, 0.4 or less, or 0.3 or less in a case where the production amount is set to 1 in a case where the corresponding wild-type microorganism or genetically modified microorganism which does not satisfy condition (I) is used.

For example, in the method of the present aspect, in a case where the genetically modified microorganism satisfying condition (I) is used, the relative production amount of succinic acid can be reduced to 0.9 or less, 0.8 or less, 0.7 or less, 0.6 or less, 0.5 or less, 0.4 or less, 0.3 or less, or 0.2 or less in a case where the production amount is set to 1 in a case where the corresponding wild-type microorganism or genetically modified microorganism which does not satisfy condition (I) is used.

In the method of the present aspect, in a case where the genetically modified microorganism satisfying condition (I) is used, the relative production amount of malic acid can be reduced to 0.95 or less, 0.9 or less, 0.89 or less, 0.88 or less, 0.8 or less, 0.7 or less, or 0.6 or less in a case where the production amount is set to 1 in a case where the corresponding wild-type microorganism or genetically modified microorganism which does not satisfy condition (I) is used.

In the method of the present aspect, in a case where the genetically modified microorganism satisfying condition (I) is used, the relative production amount of glutamic acid can be reduced to 0.9 or less, 0.8 or less, 0.7 or less, 0.6 or less, 0.5 or less, 0.4 or less, 0.3 or less, or 0.2 or less in a case where the production amount is set to 1 in a case where the corresponding wild-type microorganism or genetically modified microorganism which does not satisfy condition (I) is used.

In the method of the present aspect, in a case where the genetically modified microorganism satisfying condition (I) is used, the relative production amount of alanine can be reduced to 0.95 or less, 0.9 or less, 0.89 or less, 0.88 or less, 0.87 or less, 0.85 or less, 0.84 or less, 0.8 or less, 0.7 or less, or 0.6 or less in a case where the production amount is set to 1 in a case where the corresponding wild-type microorganism or genetically modified microorganism which does not satisfy condition (I) is used.

In the method according to the present aspect, as described above, since the production amount of by-products can be reduced, the content of impurities to be mixed with aspartic acid or a derivative thereof can be reduced. Therefore, the aspartic acid or a derivative thereof obtained by the method according to the present aspect can be suitably used as an industrial raw material.

The use of aspartic acid or a derivative thereof obtained by the method according to the present aspect is not particularly limited, and examples thereof include pharmaceutical use, food use, industrial use, fuel use, and cosmetic use. The aspartic acid or a derivative thereof may be a substance actually used for various uses or may be an intermediate raw material for producing a final product.

Hereinabove, the specific embodiments of the present invention have been described in detail, but the present invention is not limited to the above-mentioned embodiments. Various modifications, alterations, and combinations may be adopted for the configurations, elements, and characteristics without departing from the spirit of the present invention. The present invention is not limited by the above description, but only by the scope of the appended claims.

The terms “including” and “having” are each not intended to exclude the presence of elements other than the elements mentioned as the object of these terms, unless otherwise specified, and these terms are used together.

A protein, a nucleic acid, a vector, a cell, and a microorganism may be isolated. The term “isolated” means a state of being separated from a natural state or other components. An “isolated” component does not substantially include other components. The term “does not substantially include other components” means that the content of other components included in the isolated component is negligible. The content of other components included in the isolated component is, for example, 10% by mass or less, 5% by mass or less, 4% by mass or less, 3% by mass or less, 2% by mass or less, 1% by mass or less, 0.5% by mass or less, or 0.1% by mass or less. The proteins, nucleic acids, vectors, cells, and microorganisms described in the present specification can be isolated proteins, isolated nucleic acids, isolated vectors, isolated cells, and isolated microorganisms.

The contents of each document referred to in the present specification are incorporated herein by reference as a part of the present specification.

### EXAMPLES

Hereinafter, the present invention will be described with reference to Examples, but the present invention is not limited to the following Examples.

<Production of Strain with Reduced Citrate Synthase Activity>

A strain having reduced citrate synthase activity was produced using GES524/pGE333 strain (WO2020/208842A) prepared from Corynebacterium glutamicum ATCC13032 as a starting material. The GES524/pGE333 strain has a mutant ppc gene (ppc^{fbr}) having an amino acid substitution by a combination of D299N/K813S and is deficient in three genes of an ldh gene, an sdhCAB gene, and a poxB gene (pyruvate: quinone oxidoreductase) (Corynebacterium glutamicum ATCC13032 ΔIdhΔsdhΔpoxB pcc^{fbr}).

A genome sequence (NC003450.3) of Corynebacterium glutamicum ATCC13032 strain was acquired from NCBI. From the genome sequence, the upstream region (876668 to 877667) and the downstream region (877827 to 878826) of the region (877668 to 877826) including the promoter P1 (PlgltA) of gltA were set as the homologous region. Each of the upstream region and the downstream region of the promoter P1 of gltA was amplified by PCR using the genomic DNA of the GES524/pGE333 strain as a template. The primers F1 and R1 were used for amplification of the upstream region. The primers F2 and R2 were used for amplification of the downstream region.

TABLE 13

| Primer | | SEQ ID |
|---|---|---|
| name | Sequence | NO: |
| Primer F1 | ACGACGGCCAGTGAACG | 14 |
| | TTGTGTGCCCATGCAA | |
| Primer R1 | ACATATTTGTTCGGAAG | 15 |
| | CCAATTCCCCCACAATC | |
| Primer F2 | TCCGAACAAATATGTT | 16 |
| | TGAAAG | |
| Primer R2 | GGTACCGAGCTCGAATT | 17 |
| | GACGATTGCTGCACGTG | |

DNA fragments in the upstream region and the downstream region of PlgltA were linked to a plasmid pGE015 linearized by restriction enzyme treatment with EcoRI using an In-Fusion cloning kit (Takara Bio), thereby being cloned. The plasmid obtained in this manner was named plasmid ΔP1gltA.

The plasmid pGE015 is a plasmid in which a sacB gene is inserted into a BamHI/HindIII site of a plasmid pHSG299 (Takara Bio) (WO2020/208842A). The plasmid pGE015 has a kanamycin-resistant gene and a sacB gene.

The plasmid ΔP1gltA was transferred into the GES524/pGE333 strain by an electric pulse method (2500 V, 25 μF, 200 Ω; Van der Rest et al. Appl. Microbiol. Biotechnol. 52, pp. 541-545, 1999). The sample after the electric pulse method was applied on an A agar medium containing 25 μg/ml of kanamycin (composition in 1 L of the culture medium: urea: 2 g, (NH₄)₂SO₄: 7 g, KH₂PO₄: 0.5 g, K₂HPO₄: 0.5 g, MgSO₄·7H₂O: 0.5 μg, FeSO₄·7H₂O: 6 mg, MnSO₄·nH₂O: 4.2 mg, D-biotin: 200 μg, thiamine hydrochloride: 200 μg, yeast extract: 2 g, casamino acid: 7 g, glucose: 20 g, and agar: 16 g dissolved in 1,000 ml of distilled water (pH 6.6)), and cultured by a conventional method.

Since the plasmid ΔP1gltA has a kanamycin-resistant gene as a drug resistance marker, the growth strain that proliferated on the A-agar medium containing kanamycin is a strain in which the plasmid ΔP1gltA has undergone homologous recombination at one point with PlgltA on the chromosome and is incorporated into the genomic DNA together with the plasmid (see FIG. 2).

The acquired growth strain was applied on an LB agar medium (composition in 1 L of the culture medium: Bacto peptone: 10 g, yeast extract: 5 g, sodium chloride: 10 g, and agar: 16 g) to which 10% sucrose was added, and cultured by a conventional method. The transformant holding the SacB gene derived from the plasmid ΔP1gltA cannot survive in a culture medium to which sucrose is added because a toxic substance is produced. On the other hand, since the transformant from which the region derived from the plasmid containing the sacB gene has been lost by the second homologous recombination can survive even in a medium to which sucrose is added, a transformant from which the region derived from the plasmid has been lost and PlgltA has been deleted can be obtained as a growth strain. The transformant from which the entire plasmid region in the form of the plasmid ΔP1gltA falls out in the case of the second homologous recombination returns to the form of the GES524/pGE333 strain that holds the P1 promoter of the gltA gene to be intact.

The P1gltA-deficient strain was screened from the bacterial cell colonies acquired as the growth strain on the LB agar medium as described above by the colony PCR method using the primer F1 and the primer R2.

Since the primer F1 and the primer R2 are each primers designed for the 5′-end region of about 1,000 bp upstream of PlgltA and the 3′-end region of about 1,000 bp downstream of PlgltA, it is expected that DNA fragments of about 2 kb can be obtained in a case where the strain is a strain in which PlgltA is deleted. Therefore, an agarose electrophoresis method (Molecular Cloning, Sambrook et al., 1989, Cold Spring Harbor Laboratory Press) was performed on the DNA fragments obtained by the colony PCR, and a colony bacterium cell in which the deletion of PlgltA was confirmed was acquired as a PlgltA-deficient strain.

<Production of Aspartic Acid with PlgltA-Deficient Strain>

### (Culture of Bacterial Cells)

The P1gltA-deficient strain or the GES524/pGE333 strain was picked from a glycerol stock at −80° C., and plated on a plate (1.5% agar) of an A medium (composition of 1 L: urea: 2 g, (NH₄)₂SO₄: 7 g, KH₂PO₄: 0.5 g, K₂HPO₄: 0.5 g, MgSO₄·7H₂O: 0.5 g, FeSO₄·7H₂O: 6 mg, MnSO₄ nH₂O: 4.2 mg, D-biotin: 200 μg: 7 g, thiamine hydrochloride: 200 μg, yeast extract: 2 g, casamino acid: 7 g, and glucose: 20 g) and cultured overnight in a culture vessel (Panasonic MIR-162PJ) at 33° C. The bacterial cells were scraped off from the plate, inoculated into a test tube containing 10 ml of A culture medium, and cultured at 33° C. and 200 rpm for 12 hours using a shaking culture vessel (TITEC BR-43FL). After the culture, the entire amount of bacterial cells in the test tube was transferred to a 500 ml triangular flask containing 100 ml of the A culture medium and further cultured at 33° C. and 200 rpm for 12 hours using a shaking culture vessel (TITEC BR-43FL).

The main culture of the bacterial cells was performed using a 10-L jar (BMS-10NP4, ABLE). 6,200 ml of water, 800 ml of molasses (Hokkaido Sugar Co., Ltd.), 7 g of KH₂PO₄, 5 g of MgSO₄·7H₂O, and 10 ml of an antifoaming agent (CB-442, NOF Corporation) were put into a vessel and sterilized using an autoclave. Then, the entire amount of bacterial cells cultured in 100 ml of the A culture medium and 2 ml of a 50 mg/ml kanamycin solution were added thereto, and the culture was started. The conditions for the jar culture were as follows.
Temperature: 35° C.
PH: 7.0
Stirring: 600 rpm
Aeration: 5 L/minute
pH-adjusting liquid: 16N NH₃

The culture was performed for 20 to 24 hours and terminated at a stage where OD₆₁₀=60 was reached. The culture medium components were removed by centrifugation (Beckman Coulter Avanti J-26S) to obtain bacterial cells. The bacterial cells were suspended in 200 ml of water, and water was further added thereto such that the total amount of the bacterial cell suspension reached 1 L.

### (Production of Aspartic Acid)

Using the bacterial cell suspension prepared as described above, the following reaction liquid was prepared, and aspartic acid was produced.
Bacterial cell suspension: 50 ml
50% (w/v) Glucose: 10 ml
2M (NH₄)₂SO₄: 10 ml
50 mg/ml Chloramphenicol: 25 μl
Water: 30 ml

In a case where the reaction liquid was prepared as described above, the turbidity of the bacterial cells in the reaction liquid was about OD₆₁₀=200 to 250. The reaction was performed under the following conditions using Bio Jr. 8 (ABLE). 2 M (NH₄)₂CO₃ was used as the pH-adjusting liquid.
Temperature: 21° C.
pH: 8.0
Stirring: 400 rpm
Aeration: 5 ml/minute

The oxidation-reduction potential of the reaction liquid was measured at the time of reaction start (0 hours) and 16 hours and 24 hours after the reaction start. As a result, the oxidation-reduction potential of the reaction liquid was-398 to −335 mV. It was considered that the dissolved oxygen concentration in the reaction liquid during the reaction period was maintained at 0.5 mg/L or less.

16 hours and 24 hours after the reaction was started, 0.5 ml of the reaction liquid was collected and centrifuged to recover the supernatant. The glucose concentration, the amino acid concentration, and the organic acid concentration of the recovered supernatant were measured. The glucose concentration was measured using a biosensor (BF-7, manufactured by Oji Scientific Instruments Co., Ltd.). The amino acid was measured using an amino acid analysis system Prominence (Shimadzu). The organic acid was measured using an HPLC system of Prominence (Shimadzu) and a column of TSKgel OApak-A (TOSOH).

### (Results)

The results are shown in Tables 14 to 17. Table 14 shows the production amount of aspartic acid. Table 15 shows the yield of aspartic acid per 1 g of glucose. Table 16 shows the production amount of by-products. Table 17 shows the production amount of by-products per 1 g of glucose.

TABLE 14

| | Aspartic acid (g/L) |
|---|---|
| GES524/pGE333 | 11.0 |
| P1gltA-deficient strain | 13.0 |

TABLE 15

| | Aspartic acid (g/g glucose) |
|---|---|
| GES524/pGE333 | 43.6 |
| P1gltA-deficient strain | 45.5 |

TABLE 16

| | Lactic | Succinic | Malic | Glutamic | |
|---|---|---|---|---|---|
| | acid | acid | acid | acid | Alanine |
| GES524/pGE333 | 0.9 | 3.7 | 4.8 | 0.8 | 3.6 |
| P1gltA-deficient strain | 0.3 | 0.4 | 4.2 | 0.2 | 3.0 |

(g/L)

TABLE 17

| | Lactic | Succinic | Malic | Glutamic | |
|---|---|---|---|---|---|
| | acid | acid | acid | acid | Alanine |
| GES524/pGE333 | 2.2 | 5.6 | 15.6 | 2.8 | 10.7 |
| P1gltA-deficient strain | <0.05 | 1.2 | 14.1 | 0.6 | 5.0 |

(g/g glucose)

From the results shown in Tables 14 and 15, it was confirmed that the production amount and the yield of aspartic acid were increased in the PlgltA-deficient strain compared with the PlgltA-non-deficient strain (GES524/pGE333 strain). In addition, from the results shown in Tables 16 and 17, it was confirmed that the production amount and the yield of by-products were reduced in the PlgltA-deficient strain compared with the PlgltA-non-deficient strain (GES524/pGE333 strain).

It is expected that the suppression of GltA activity suppresses the supply of the carbon source to the TCA circuit, and thus the production amount of lactic acid or alanine derived from pyruvic acid upstream thereof is generally increased. In fact, since the production amount and the yield of succinic acid and glutamic acid, which are metabolites of the TCA cycle produced through GltA, were significantly reduced (Tables 16 and 17), it was confirmed that the supply of the carbon source to the TCA cycle was inhibited by the suppression of the GltA activity. Interestingly, in the P1gltA-deficient strain, the production amount and the yield of lactic acid and alanine were reduced compared with the non-deficient strain (GGS524/pGE333 strain) (Tables 16 and 17). This is a result different from the expectation, and there is almost no prior art describing such an effect.

<Production of Strain with Reduced Oxaloacetate Decarboxylase Activity>

A strain having reduced oxaloacetate decarboxylase activity was produced using GES524/pGE333 strain (WO2020/208842A) prepared from Corynebacterium glutamicum ATCC13032 as a starting material.

From the genome sequence (NC003450.3) of Corynebacterium glutamicum ATCC13032 strain, the upstream region and the downstream region of the region (1358259 to 1359065) including odx were set as the homologous region. Each of the upstream region and the downstream region of the odx was amplified by PCR using the genomic DNA of the GES524/pGE333 strain as a template.

DNA fragments in the upstream region and the downstream region of odx were linked to a plasmid pGE015 linearized by restriction enzyme treatment with EcoRI using an In-Fusion cloning kit (Takara Bio), thereby being cloned. The plasmid obtained in this manner was named plasmid Δodx.

The plasmid Δodx was transferred into the GES524/pGE333 strain by an electric pulse method (2500 V, 25 μF, 200 52; Van der Rest et al. Appl. Microbiol. Biotechnol. 52, pp. 541-545, 1999). The sample after the electric pulse method was applied on an A agar culture medium containing 25 μg/ml of kanamycin and cultured by a conventional method.

Since the plasmid Δodx has a kanamycin-resistant gene as a drug resistance marker, the growth strain proliferated on the A-agar medium containing kanamycin is a strain in which the plasmid Δodx has undergone homologous recombination at one point with odx on the chromosome and is incorporated into the genomic DNA together with the plasmid. The growth strain acquired in this manner was applied on an LB agar medium to which 10% sucrose was added and cultured by a conventional method. From the bacterial cell colonies acquired as the growth strain on the LB agar medium, an odx-deficient strain was screened by a colony PCR method.

### <Production of Aspartic Acid by Oxd-Deficient Strain>

The culture of the bacterial cells and the production of aspartic acid were performed in the same manner as in the <Production of aspartic acid with PlgltA-deficient strain>except that the odx-deficient strain was used instead of the P1gltA-deficient strain. It was confirmed that the production amount and the yield of aspartic acid were increased in the odx-deficient strain compared with the odx-non-deficient strain (GES524/pGE333 strain). It was confirmed that the production amount and the yield of alanine were reduced in the odx-deficient strain compared with the odx-non-deficient strain (GES524/pGE333 strain).

1. A genetically modified microorganism which satisfies the following condition (I) and at least one condition selected from the group consisting of the following conditions (III), (IV) and (VI),
condition (I): citrate synthase activity is reduced or inactivated compared with a wild-type microorganism corresponding to the genetically modified microorganism,
condition (III): succinate dehydrogenase activity or fumarate reductase activity is reduced or inactivated compared with the wild-type microorganism,
condition (IV): lactate dehydrogenase activity is reduced or inactivated compared with the wild-type microorganism,
condition (VI): pyruvate: quinone oxidoreductase activity is reduced or inactivated compared with the wild-type microorganism.

2. The genetically modified microorganism according to claim 1,
wherein the genetically modified microorganism further satisfies the following condition (V),
condition (V): modified phosphoenolpyruvate carboxylase activity that exhibits resistance to feedback inhibition by aspartic acid in wild-type phosphoenolpyruvate carboxylase activity, or exogenous phosphoenolpyruvate carboxylase activity that exhibits higher resistance to feedback inhibition by aspartic acid compared with the wild-type phosphoenolpyruvate carboxylase activity exhibited by the wild-type microorganism is provided.

3. The genetically modified microorganism according to claim 1,
wherein the genetically modified microorganism further satisfies the condition (II) (,
condition (II): oxaloacetate decarboxylase activity is reduced or inactivated compared with the wild-type microorganism.

4. The genetically modified microorganism according to claim 1,
wherein the genetically modified microorganism belongs to a Gram-positive bacterium.

5. The genetically modified microorganism according to claim 1,
wherein the genetically modified microorganism is used for producing aspartic acid or a derivative of aspartic acid.

6. The genetically modified microorganism according to claim 5,
wherein a production amount of at least one by-product selected from the group consisting of amino acids other than the aspartic acid and organic acids is reduced compared with a microorganism not satisfying the condition (I).

7. The method for producing aspartic acid or a derivative of aspartic acid, the method comprising:
(p) producing aspartic acid or a derivative of aspartic acid using a cell of the genetically modified microorganism according to claim 1 or a treated product of the cell; and
(q) recovering the aspartic acid or the derivative of aspartic acid.

8. The method for producing aspartic acid or a derivative of aspartic acid according to claim 7,
wherein (p) is performed in a reaction medium having a dissolved oxygen concentration of 0.5 mg/L or less.

9. The method for producing aspartic acid or a derivative of aspartic acid according to claim 7,
wherein a production amount of at least one by-product selected from the group consisting of amino acids other than the aspartic acid and organic acids is reduced compared with a case of producing the aspartic acid using a cell of a microorganism not satisfying the condition (I) or a treated product of the cell.
